(19) United States
(12) Patent Application Publication
(10) Pub. No.: US 2025/0051283 A1
(43) Pub. Date: Feb. 13, 2025

(54) THERAPEUTIC COMPOUNDS AND METHODS

(71) Applicant: RUTGERS, THE STATE UNIVERSITY OF NEW JERSEY, New Brunswick, NJ (US)

(72) Inventor: Jun Wang, New Brunswick, NJ (US)

(73) Assignee: RUTGERS, THE STATE UNIVERSITY OF NEW JERSEY, New Brunswick, NJ (US)

(21) Appl. No.: 18/790,041

(22) Filed: Jul. 31, 2024

Related U.S. Application Data
(60) Provisional application No. 63/530,433, filed on Aug. 2, 2023.

(51) Int. Cl. C07D 209/52; A61K 31/403; A61K 31/4045; A61K 31/428; A61P 31/14; C07D 409/12; C07D 417/12

(52) U.S. Cl. CPC C07D 209/52; A61K 31/403; A61K 31/4045; A61K 31/428; A61P 31/14; C07D 409/12; C07D 417/12

(57) ABSTRACT

The invention provides a compound of formula I.

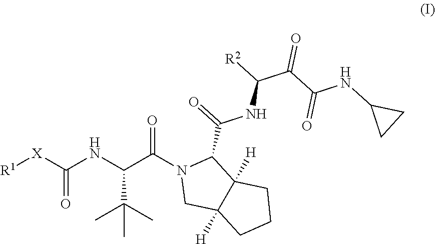

or a salt thereof, wherein R¹—R² have any of the values described in the specification, as well as compositions comprising a compound of formula I. The compounds are useful as antiviral agents.

### CROSS-REFERENCE TO RELATED APPLICATION

This application claims priority to U.S. Provisional Application No. 63/530,433 that was filed on Aug. 2, 2023. The entire content of the applications referenced above is hereby incorporated by reference herein.

### STATEMENT REGARDING FEDERALLY SPONSORED RESEARCH

This invention was made with government support under AI157046 and AI147325 awarded by the National Institutes of Health. The government has certain rights in the invention.

### BACKGROUND

Enterovirus D68 (EV-D68) is a non-enveloped, positive-sense, and single-stranded RNA virus that causes moderate to severe respiratory illnesses in children (Cassidy, H.; et al., Front Microbiol 2018, 9, 2677; Baggen, J.; et al., Nat Rev Microbiol 2018, 16 (6), 368-381; and Sun, J.; et al., Viruses 2019, 11 (6)). EV-D68 belongs to the enterovirus genus of Picornaviridae family. The enterovirus genus also contains many other important human pathogens, including poliovirus, enterovirus A71 (EV-A71), rhinovirus, and coxsackievirus (Baggen, J.; et al., Nat Rev Microbiol 2018, 16 (6), 368-381; and Lugo, D.; Krogstad, P. Current opinion in pediatrics 2016, 28 (1), 107-113). EV-D68 has two distinct features compared to other viruses in the enterovirus genus (Royston, L.; et al., PLoS Pathog 2018, 14 (4)): 1) EV-D68 is cold-adapted and prefers a lower growth temperature (33° C.) instead of the regular body temperature (37° C.); 2) EV-D68 is acid sensitive (Liu, Y; et al., Proc. Natl. Acad. Sci. U.S.A. 2018, 115 (52), E12209-E12217), which explains why it cannot infect through the gastrointestinal tract as other enteroviruses, possibly due to the low pH in the gut.

EV-D68 was first isolated in California in 1962 and remained a neglected rare disease until 2014 (Oermann, C. M.; et al., Ann Am Thorac Soc 2015, 12 (5), 775-781). EV-D68 came to the public attention when recent outbreaks coincided with neurological complications (Cassidy, H.; et al., Front Microbiol 2018, 9, 2677; Lugo, D.; Krogstad, P. Current opinion in pediatrics 2016, 28 (1), 107-113; and Dyda, A.; et al., Euro Surveill 2018, 23 (3), 16-24). Several outbreaks occurred in the United States as well as other parts of the world since 2014, and there appears to be a biennial pattern of EV-D68 outbreak since then (Morens, D. M.; et al., mBio 2019, 10 (2); and Messacar, K.; et al., Ann Neurol 2016, 80 (3), 326-338). This virus seems to be evolving over time, leading to more severe pathogenicity (Dyda, A.; et al., Euro Surveill 2018, 23 (3), 16-24; Morens, D. M.; et al., mBio 2019, 10 (2); Messacar, K.; et al., Ann Neurol 2016, 80 (3), 326-338; Brown, D. M.; et al., mBio 2018, 9 (5); and Midgley, C. M.; et al., Lancet Respir Med 2015, 3 (11), 879-887). For example, infection with contemporary EV-D68 virus was linked to neurological complications, and children infected with EV-D68 had symptoms like muscle weakness, paralysis and even death in the severe cases (Greninger, A. L.; et al., Lancet Infect Dis 2015, 15 (6), 671-682; and Christy, A.; Messacar, K., J Child Neurol 2019, 34 (9), 511-516). Recent study has shown that contemporary EV-D68 strains (US/KY/14), but not historical EV-D68 strains (Fermon), were able to infect neuronal cells and cause cytopathic effect (Brown, D. M.; et al., mBio 2018, 9 (5)). In mouse model studies, EV-D68 could be isolated from central nervous system (CNS) tissues including spinal cord, brain, and cerebral fluid (Hixon, A. M.; et al., PLoS Pathog 2017, 13 (2), e1006199; and Morrey, J. D.; et al., Viruses 2018, 10 (1); and Hurst, B. L.; et al., Virology 2019, 526, 146-154). Although EV-D68 virus was rarely identified in the CNS tissues in human patients, there is nevertheless a strong correlation between EV-D68 infection and neurological complications as shown by a number of studies (Dyda, A.; et al., Euro Surveill 2018, 23 (3), 16-24; and Bowers, J. R.; et al., mBio 2019, 10 (1)).

Despite the severe disease outcome posed by EV-D68, there is currently no antiviral or vaccine available (Baggen, J.; et al., Nat Rev Microbiol 2018, 16 (6), 368-381), and current treatment is limited to supportive care. Several compounds were reported in the literature that inhibit EV-D68 amplification in cell culture, and many of the compounds tested were either originally developed as rhinovirus antivirals or repurposed from approved drugs (Sun, L.; et al., Antimicrobial Agents and Chemotherapy 2015, 59 (12), 7782-7785; Rhoden, E.; et al., Antimicrobial agents and chemotherapy 2015, 59 (12), 7779-7781; and Smee, D. F.; et al., Antiviral Res 2016, 131, 61-65). Unfortunately, the reported EV-D68 antivirals either have moderate potency or concerning side effects (Hurst, B. L.; et al., Virology 2019, 526, 146-154; Smee, D. F.; et al., Antiviral Res 2016, 131, 61-65; and Messacar, K.; et al., Neurology 2019, 92 (18), e2118-e2126). Therefore, it is imperative to develop potent and specific EV-D68 antivirals for the prophylaxis and treatment of EV-D68 infection (Musharrafieh, R.; et al., Journal of virology 2019, 93 (7), e02221-02218; and Musharrafieh, R.; et al., J Med Chem 2019, 62 (8), 4074-4090).

EV-D68 is a non-enveloped virus that is surrounded by viral capsid proteins (Egorova, A.; et al., European journal of medicinal chemistry 2019, 178, 606-622). The virion capsid consists of four proteins: VP1, VP2, VP3, and VP4. A concave pocket is formed in VP1 and it is important for cell receptor binding. The concave pocket is frequently referred as canyon. Small molecules such as pleconaril that are capable of binding to this canyon can stabilize the capsid protein and prevent viral uncoating, leading to inhibition of viral replication (Liu, Y.; Sheng, J.; et al., Science 2015, 347 (6217), 71-74; and Egorova, A.; et al., European journal of medicinal chemistry 2019, 178, 606-622). As such, the VP1 protein is a validated drug target.

Telaprevir is a drug approved by the FDA in 2011 for treatment of hepatitis C. In a recently published manuscript, telaprevir was identified as a potent EV-D68 inhibitor. In spite of this discovery, there remains a need for agents that are useful for the prevention and treatment of viral infection, including the prevention and treatment of EV-D68. In particular, there is a need for antiviral compounds with improved potency and/or selectivity.

Currently there is a need for EV-D68 specific capsid inhibitors that can be used alone or in combination with other antivirals to combat EV-D68 infections.

### SUMMARY

In one aspect the present invention provides compounds described herein (e.g., compounds having antiviral activity).

Accordingly, the invention provides a compound of formula (I):

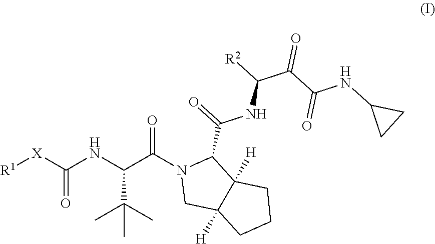

or a salt thereof wherein:
X is a bond or is (C₁-C₆)alkylene;
R¹ is (C₃-C₁₅)cycloalkyl that is optionally substituted with one or more groups independently selected from the group consisting of halo and (C₁-C₆)alkyl, which (C₁-C₆)alkyl is optionally substituted with one or more groups independently selected from the group consisting of halo; and
R² is (5-10 membered heteroaryl)(C₁-C₃)alkyl, cyclopropylmethyl, 2-methylpropyl, (C₃-C₆)cycloalkyl, isopropyl, propyl, or 1-methylpropyl.

The invention also provides a pharmaceutical composition comprising a compound of formula I or a pharmaceutically acceptable salt thereof, and a pharmaceutically acceptable excipient.

The invention also provides a method for treating or preventing a viral infection in an animal (e.g., a mammal such as a human) comprising administering a compound of formula I or a pharmaceutically acceptable salt thereof to the animal.

The invention also provides a compound of formula I or a pharmaceutically acceptable salt thereof for use in medical therapy.

The invention also provides a compound of formula I or a pharmaceutically acceptable salt thereof for the prophylactic or therapeutic treatment of a viral infection.

The invention also provides the use of a compound of formula I or a pharmaceutically acceptable salt thereof to prepare a medicament for treating a viral infection in an animal (e.g. a mammal such as a human).

The invention also provides processes and intermediates disclosed herein that are useful for preparing a compound of formula I or a salt thereof.

### BRIEF DESCRIPTION OF DRAWINGS

The patent or application file contains at least one drawing executed in color. Copies of this patent or patent application publication with color drawings will be provided by the Office upon request and payment of the necessary fee.

FIGS. 1A-1D. Crystal structures of EV-D68 2A^{pro} and EV-D68 2A^{C107A} and comparison of the crystal structures of EV-D68 2A^{pro} (7 MG0) and EV-71 2A^{pro} (4FVB). (FIG. 1A) Crystal structure derived model of EV-D68 2A^{pro} (7 MG0) molecule B. The N-terminal domain is shown in gold, while the C-terminal domain is colored cyan. The four conserved residues coordinated to the bound Zn²⁺ are labeled. (FIG. 1B) Overlay of EV-D68 2A^{pro} (gold, 7 MG0) and EV-D68 2A^{C107A} (magenta, 7JRE). Essential residues coordinating the bound Zn⁺² atom are labeled. (FIG. 1C) Ribbon diagrams showing the overlay of Ca atoms EV-D68 2A^{pro} (N-terminal domain, gold; C-terminal domain, cyan (7 MG0) and EV-71 2A^{pro} (green) (4FVB). The differences between the relative positions of the residues in the loops connecting β-strands bII2 and eII are shown by a blue dashed arrow for EV-D68 2A^{pro} and a red dashed arrow for EV-71 2A^{pro}. The molecules have been rotated ˜180° around the x- and y-axis. (FIG. 1D) The coordination of the Zn+2 cation by Cys53, Cys55, Cys113, and His115 is highly spatially conserved. All model figures were generated using Pymol.

FIG. 2. Docking pose of telaprevir produced with induced fit docking using the crystal structure of the EV-D68 2A^{pro} (PDB ID 7 MG0).

FIGS. 3A-3F. M.D. simulations of telaprevir and Jun11762 in complex with EV-D68 2A^{pro}. Covalent docked poses of telaprevir (FIG. 3A) and Jun11762 (FIG. 3D) resulted from 500 ns-MD simulations. Ligand carbons are shown in green, and protein in light blue, with important amino acid residues in grey sticks. Non-polar hydrogens were omitted for clarity. RMSD plots of Ca carbons of the protein (blue diagram) and telaprevir (FIG. 3B) and Jun11762 (FIG. 3E) (orange diagram). Stabilizing interactions inside the binding area of telaprevir (FIG. 3C) and Jun11762 (FIG. 3F); hydrogen bonding interactions bar is depicted in green, hydrophobic interactions in magenta, and water bridges in blue. They are considered important when the frequency bar is ≥0.2.

FIG. 4. Telaprevir analogs with variations at the P2, P3, P4 substitutions and the reactive warhead. Key structural variations compared to telaprevir were highlighted in red. Enzymatic inhibitory activity IC₅₀ value was determined in the FRET-based assay. Antiviral EC₅₀ value was determined in the CPE assay with EV-D68 US/MO/14-18947 in R.D. cells. Cytotoxicity CC₅₀ value was determined using the neutral red uptake method in R.D. cells. The results are the mean±standard deviation of two independent experiments with three repeats in each experiment.

FIG. 5. Telaprevir analogs with variations at the P1 substitutions and the reactive warhead. Key structural variations compared to Jun8861 were highlighted in red. Enzymatic inhibitory activity IC₅₀ value was determined in the FRET-based assay. Antiviral EC₅₀ value was determined in the CPE assay with EV-D68 US/MO/14-18947 in R.D. cells. Cytotoxicity CC₅₀ value was determined using the neutral red uptake method in R.D. cells. The results are the mean±standard deviation of two independent experiments with three repeats in each experiment.

FIGS. 6A-6C. Plaque assay results of telaprevir analogs in inhibiting EV-D68 US/MO/14-18947. (FIG. 6A) Plaque images. The images are representatives of two repeats. (FIG. 6B) Curve fitting. (FIG. 6C) EC₅₀ values. The results are fthe mean±standard deviation of two repeats.

FIG. 7. Summary of the structure-activity relationships of telaprevir in inhibiting EV-D68 2A^{pro}.

### DETAILED DESCRIPTION

The following definitions are used, unless otherwise described: halo or halogen is fluoro, chloro, bromo, or iodo. Alkyl, alkoxy, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and and higher homologs and isomers.

The term “cycloalkyl” refers to a saturated or partially unsaturated (non-aromatic) all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocycles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocycles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from cycloalkyl, aryl, heterocycle, and heteroaryl. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, triazolyl, tetrazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, and quinazolyl.

As used herein, the term “heteroatom” is meant to include oxygen (O), nitrogen (N), sulfur (S) and silicon (Si).

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylen-oxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfanylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” includes mammals, fish, amphibians, reptiles, birds and invertebrates. The term “mammal” includes humans, higher non-human primates, rodents, domestic, cows, horses, pigs, sheep, dogs and cats. In one embodiment, the animal is a mammal. In one embodiment, the animal is a human. The term “patient” as used herein refers to any animal including mammals. In one embodiment, the patient is a mammalian patient. In one embodiment, the patient is a human patient.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (2H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; and (C₃-C₆)cycloalkyl can be cyclopropyl, cyclobutyl, cyclopentyl, or cyclohexyl.

A specific value for R¹ is (C₆-C₁₀)cycloalkyl that is optionally substituted with one or more groups independently selected from the group consisting of halo and (C₁-C₆)alkyl, which (C₁-C₆)alkyl is optionally substituted with one or more groups independently selected from the group consisting of halo.

A specific value for R¹ is (C₆-C₁₀)cycloalkyl.

A specific value for R¹ is adamantyl that is optionally substituted with one or more groups independently selected from the group consisting of halo and (C₁-C₆)alkyl, which (C₁-C₆)alkyl is optionally substituted with one or more groups independently selected from the group consisting of halo.

A specific value for R¹ is adamantyl.

A specific value for R² is (5-membered heteroaryl)(C₁-C₃)alkyl.

A specific value for R² is (9-membered heteroaryl)(C₁-C₃)alkyl.

A specific value for R² is (5-membered heteroaryl)methyl.

A specific value for R² is (9-membered heteroaryl)methyl.

A specific value for R² is cyclopropylmethyl.

A specific value for R² is 2-methylpropyl.

A specific value for R² is (C₃-C₆)cycloalkyl.

A specific value for R² is cyclopentyl.

A specific value for R² is cyclohexyl.

A specific value for R² is isopropyl.

A specific value for R² is propyl.

A specific value for R² is 1-methylpropyl.

A specific value for X is methylene.

A specific compound or salt is selected from:

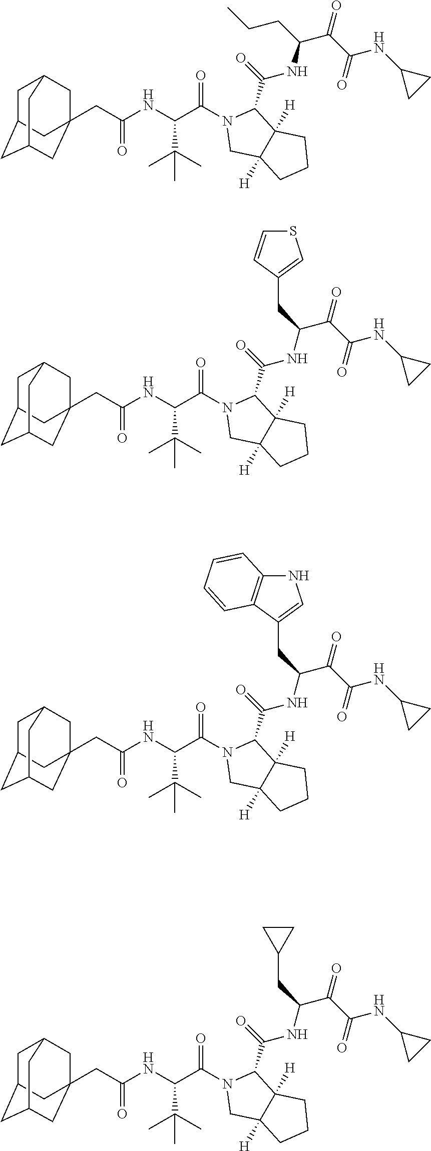 [SMILES: CCC[C@H](NC(=O)[C@@H]1[C@H]2CCC[C@H]2CN1C(=O)[C@@H](NC(=O)CC12CC3CC(CC(C3)C1)C2)C(C)(C)C)C(=O)C(=O)NC1CC1 ; CC(C)(C)[C@H](NC(=O)CC12CC3CC(CC(C3)C1)C2)C(=O)N1C[C@@H]2CCC[C@@H]2[C@H]1C(=O)N[C@@H](Cc1ccsc1)C(=O)C(=O)NC1CC1 ; CC(C)(C)[C@H](NC(=O)CC12CC3CC(CC(C3)C1)C2)C(=O)N1C[C@@H]2CCC[C@@H]2[C@H]1C(=O)N[C@@H](Cc1c[nH]c2ccccc12)C(=O)C(=O)NC1CC1 ; CC(C)(C)[C@H](NC(=O)CC12CC3CC(CC(C3)C1)C2)C(=O)N1C[C@@H]2CCC[C@@H]2[C@H]1C(=O)N[C@@H](CC1CC1)C(=O)C(=O)NC1CC1]

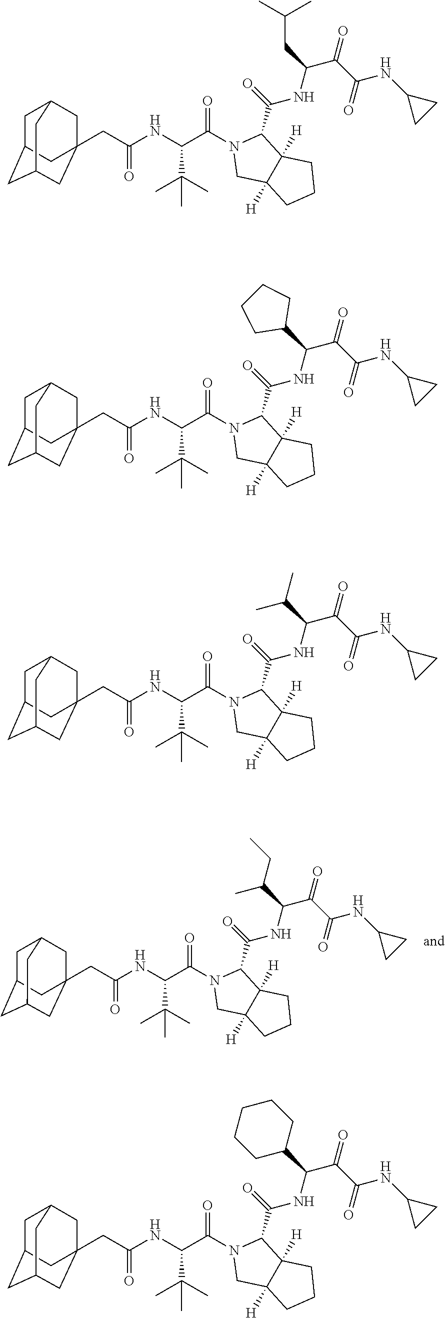

and salts thereof.

Processes for preparing compounds of formula I are provided as further embodiments of the invention and are illustrated by the following procedures in which the meanings of the generic radicals are as given above unless otherwise qualified.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful for the treatment of viral infections (e.g., protease inhibitors). Examples of such agents include, rupintrivir, AG7404, telaprevir, and fluoxetine. Accordingly, in one embodiment the invention also provides a composition comprising a compound of formula I, or a pharmaceutically acceptable salt thereof, at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to an animal to treat a viral infection.

The invention will now be illustrated by the following non-limiting Examples.

### EXAMPLES

### Example 1. X-Ray Crystal Structures of EV-D68 2A^{pro} and 2A^{C107A}

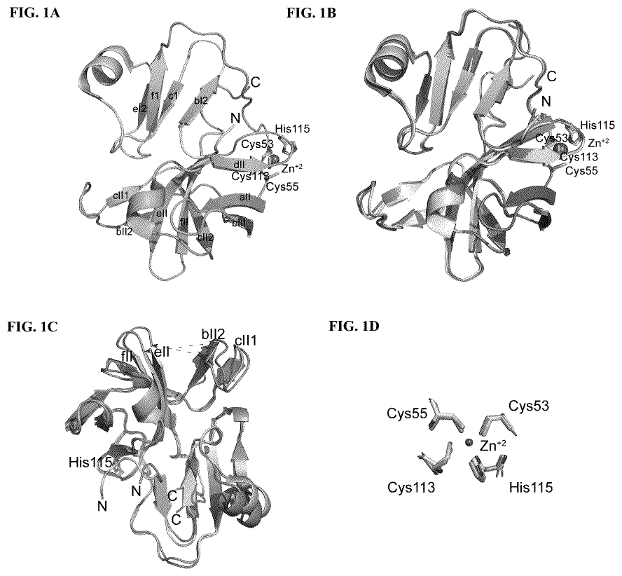

The asymmetric unit contains six EV-D68 2A^{pro} molecules (A-F). The overall structures of the six molecules demonstrate high similarity with r.m.s.d. values ranging from 0.27-0.41 Å over 109-116 Cα atoms. EV-D68 2A^{pro} adopts the classical 2A protease fold consisting of two domains. The N-terminal domain is comprised of a four-stranded sheet (bI2, cI, eI2, and fI) and two orthogonally oriented β-sheets (aII, bII, cII2, dII, eII, and fII) with a tightly bound zinc atom C-terminal domain (FIG. 1A). Four conserved residues, Cys53, Cys55, Cys113, and His115, comprise the zinc ion-binding site (FIG. 1A). Mutation of C107A does not significantly alter the overall structure of EV-D68 2A^{C107A} with an r.m.s.d. of 0.43 Å over 134 Cα atoms (molecule B of EV-D68 2A^{pro} compared to molecule E of EV-D68 2A^{C107A}) (FIG. 1).

The structures of EV-D68 2A^{pro} (7 MG0) and EV-71 2A^{pro} (4FVB) are overall very similar with an r.m.s.d. of 0.74 Å over 116 Ca atoms. However, closer inspection finds loop eII-fII and loop bII2-cII1 have shifted in the apo EV-D68 2A^{pro} structure, widening the cavity between the loops from 10.1 Å (Val81 Ca to Gly 128 Ca, red dashed line) to 13.8 Å (Ile81 Ca to Gly 125 Ca, blue dashed line) (FIG. 1C). The positions of the residues coordinating the Zn⁺² cation binding are highly conserved (FIG. 1D).

### Example 2. Molecular Dynamics Simulations of Telaprevir in Complex with EV-D68 2A^{pro}

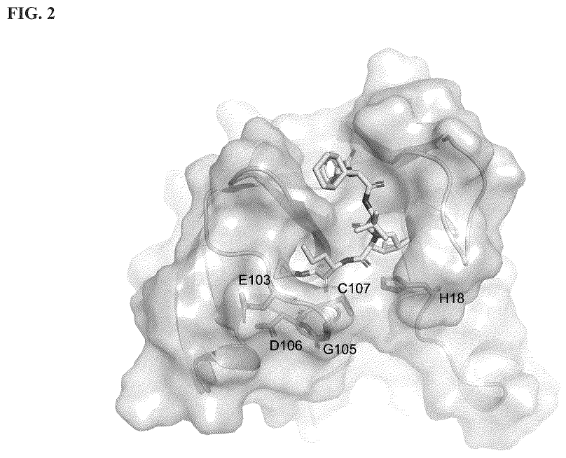

To gain insights into the binding pose of telaprevir in complex with EV-D68 2A^{pro}, we produced with induced fit docking using the structure of the EV-D68 2A^{pro} apo protein (PDB ID 7 MG0), and the docking pose of telaprevir is shown in FIG. 2. In the telaprevir complex, the ketoamide warhead was trapped in the H18-D106-C107 catalytic triad. The ketoamide oxygen sat in the oxyanion hole and can interact with close backbone amide nitrogens of protease residues G105 and D106. Meanwhile, the NP nitrogen of H18 acts as the general base in the catalytic triad. In this docking pose, the C107 thiol is poised to form a covalent bond with the ketone from telaprevir.

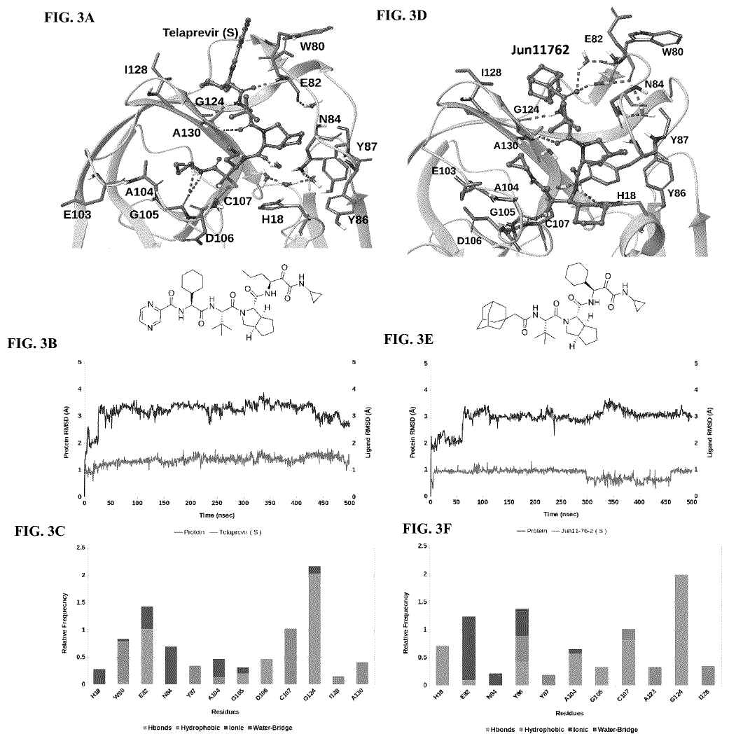

A covalently bound conformation with telaprevir in which a covalent bond was formed between the ketoamide warhead and the catalytic cysteine 107 was then generated. An (S) configuration was generated, which is typical for most protease inhibitors, although the (R) configuration has also been observed. A 500 ns-MD simulation was performed to optimize steric clashes between the ligand and the binding pocket and investigate possible conformational rearrangements. The final snapshot of the binding complex is shown in FIG. 3A. The MD simulations showed that the 2A^{pro} conformation was stabilized after ˜50 ns while telaprevir retained the docking conformation during the trajectory (FIG. 3B).

Most hydrogen bonding interactions observed in the docking pose in the non-covalent complex (FIG. 2) were also observed in the covalent complex (FIGS. 3A, C). In particular, telaprevir formed three hydrogen bonds with the protease backbone: (1) the P1 ketoamide nitrogen with the carbonyl oxygen of G105 and D106, (2) the P2 carbonyl oxygen with the amide nitrogen of G124, and (3) the P4 amide carbonyl oxygen with the amide nitrogen of E82 (FIGS. 3A, C). As shown in FIG. 3A, the hydrogen bond observed in the non-covalent complex between N84 carbonyl amide side chain and P2 amide nitrogen was disrupted by waters that enter this area concentrated between the H18 imidazole, N84 amide side chain, and C107. Indeed, in FIG. 3B waters bridges are shown between H18 and N84. Hydrophobic interactions were formed between W80 and P5 pyrazine ring, Y87 and P2 lactam cyclopentyl, C107 and P1 propyl, and P4 cyclohexyl and A130 (FIG. 3).

Similarly M.D. simulations were performed to gain insights into the binding mode of Jun11762. The 500 ns-MD simulations with Jun11762 complex are shown in FIGS. 3D, E, and F. The MD simulations showed that the 2A^{pro} conformation was stabilized after ˜60 ns while Jun11762 retained the docking conformation during the trajectory (FIG. 3E). Jun11762 binds similarly to telaprevir except for the additional hydrogen bond between P1 amide N.H. and the H18 side chain imidazole (FIGS. 3D, F). The adamantyl group also contributes to more significant hydrophobic interactions with 1128 (FIG. 3F vs 3C).

### Example 3. Exploring P2, P3, P4 Substitutions and the Reactive Warhead

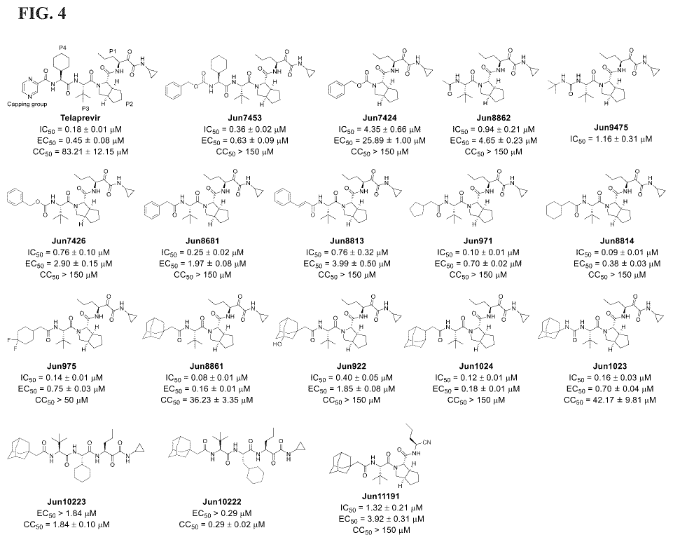

Guided by the docking pose of telaprevir with EV-D68 2A^{pro} (FIG. 3A), structure-based lead optimization was performed. Telaprevir inhibited EV-D68 2A^{pro} and EV-D68 US/MO/14-18947 strain with IC₅₀ of 0.18 μM and EC₅₀ of 0.45 μM in the FRET enzymatic assay and CPE assay, respectively, and was included as a control. Replacing the pyrazine capping group in telaprevir with carboxybenzyl (Cbz) led to compound Jun7453 with slightly decreased antiviral potency (EC₅₀=0.63 μM vs 0.45 μM) (FIG. 4). To explore the importance of the P3 tert-butyl and P4 cyclohexyl substitutions in 2A^{pro} inhibition, compounds Jun7424 and Jun8862 were designed. Compound Jun7424 with deletions in the P3 and P4 substitutions had drastically reduced enzymatic inhibition (IC₅₀=4.35 μM vs 0.18 μM) and antiviral activity (EC₅₀=25.89 μM vs 0.45 μM). Compound Jun8862 with a deletion in the P4 substitution also had reduced enzymatic inhibition (IC₅₀=0.94 μM vs 0.18 μM) and antiviral activity (EC₅₀=4.65 μM vs 0.45 μM). These results suggest that P3 and P4 substitutions are essential for 2A^{pro} inhibition and antiviral activity. The SAR is consistent with the binding pose of telaprevir in 2A^{pro} predicted by the M.D. simulations, which revealed that the P3 tert-butyl and P4 cyclohexyl substitutions form hydrophobic interactions with A130 and 1128, respectively (FIG. 3A).

Next, a series of compounds were designed with various aromatic and hydrophobic substitutions as mimetics of the P4 cyclohexyl substitution in telaprevir. Jun9475 with the tert-butyl urea capping group had reduced enzymatic inhibition (IC₅₀=1.16 μM vs 0.18 μM). Compounds Jun7426, Jun8681, and Jun8813 with aromatic capping groups at P4 position all had reduced enzymatic inhibition and antiviral activity compared to telaprevir. These results suggest that aromatic substitutions were not preferred at the P4 site. As the P4 cyclohexyl substitution at the P4 site of telaprevir forms hydrophobic interactions with 1128 (FIG. 3A), we hypothesized that aliphatic hydrophobic substitutions might be favored at this subsite. Indeed, compounds Jun971 (cyclopentyl), Jun8814 (cyclohexyl), Jun975 (difluorocyclohexyl), and Jun8861 (adamantyl) all had comparable or improved enzymatic inhibition (IC₅₀=0.08 to 0.14 μM) and antiviral activity (EC₅₀=0.16 to 0.75 μM) compared to telaprevir. The most potent compound Jun8861 inhibited EV-D68 2A^{pro} with an IC₅₀ of 0.08 μM and the EV-D68 US/MO/14-18947 virus with an EC₅₀ of 0.16 μM. Compound Jun922 with the 3-hydroxyadamantyl substitution had reduced antiviral activity compared to the adamantyl analog Jun8861 (EC₅₀=1.85 μM vs 0.16 μM), suggesting hydrophilic substitution is not preferred. Compound 1024 with 2-adamantyl substitution had similar antiviral potency as Jun8861 (EC₅₀=0.18 μM vs 0.16 μM). Replacing the amide linker in Jun1024 with a urea link led to compound Jun1023 with reduced enzymatic inhibition and antiviral activity (EC₅₀=0.70 μM vs 0.16 μM) compared to Jun8861.

Compounds Jun10223 and Jun10222 were designed to explore the importance of the P2 cyclopentylproline substitution in telaprevir. However, both compounds were highly cytotoxic (CC₅₀<2 μM) and were not further pursued.

Inspired by nirmatrelvir, which contains a nitrile reactive warhead, the telaprevir analog Jun11191 with the nitrile warhead was designed. However, Jun11191 had a significantly reduced enzymatic inhibition (IC₅₀=1.32 vs 0.08 μM) and antiviral activity (EC₅₀=3.92 vs 0.16 μM) compared to Jun8861.

In summary, SAR results showed that the P2 cyclopentylproline, P3 tert-butyl, and the α-ketoamide reactive warhead in telaprevir are preferred for 2A^{pro} inhibition, and aliphatic hydrophobic substitutions are preferred at the P4 position. The SAR results are consistent with the binding mode predicted by the M.D. simulations (FIG. 3).

### Example 4. Exploring P1 Substitution and the Reactive Warhead

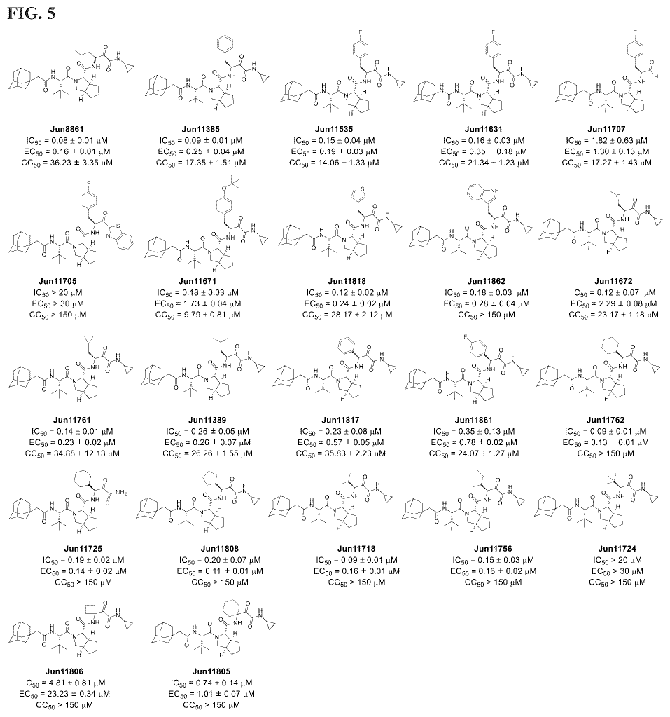

Starting from the most potent lead compound Jun8861, the P1 substitution were explored to further improve the antiviral potency (FIG. 5). According to the binding pose predicted by the M.D. simulations, the P1 norvaline substitution in telaprevir forms hydrophobic interactions with A104 and intramolecular hydrophobic interactions with the P3 tert-butyl substitution (FIG. 3A). Therefore, it was hypothesized that other hydrophobic substitutions, including aromatic and aliphatic ones, might be tolerated at the P1 position. Compound Jun11385 with benzyl substitution at P1 position had similar enzymatic inhibition (IC₅₀=0.09 vs 0.08 μM), antiviral activity (EC₅₀=0.25 vs 0.16 μM), but increased cytotoxicity (CC₅₀=17.35 vs 36.23 μM) compared to Jun8861. Compound Jun11763 with the 4-fluorobenzyl substitution had comparable enzymatic inhibition and antiviral activity as Jun11385. Replacing the amide linker in Jun11763 with a urea linker led to compound Jun11631 with similar enzymatic inhibition (IC₅₀=0.16 vs 0.15 μM) and but reduced antiviral activity (EC₅₀=0.35 μM vs 0.19 μM). Next, the reactive warhead was examined. Replacing the α-ketoamide with aldehyde led to compound Jun11707 with reduced enzymatic inhibition (IC₅₀=1.82 vs 0.15 μM) and antiviral activity (EC₅₀=1.30 μM vs 0.19 μM). Compound Jun11705 with the ketobenzothiazole warhead was inactive (IC₅₀>20 μM). Compound Jun11671 with the 4-(tert-butoxy)phenyl substitution showed reduced enzymatic inhibition (IC₅₀=0.18 vs 0.09 μM) and antiviral activity (EC₅₀=1.73 μM vs 0.25 μM) compared to Jun11385, suggesting bulky substitutions are not preferred at P1 position. Jun11818 and Jun11862 with the thienyl and indole substitutions at P1 position had comparable enzymatic inhibition and antiviral activity as Jun11385. Notably, Jun11862 was not cytotoxic (CC₅₀>150 μM). Jun11672 with the methoxymethyl P1 substitution was not active as Jun8861 with the propyl substitution, suggesting hydrophilic substitution is not preferred at P1 position. Compounds Jun11761 and Jun11389 with the cyclopropylmethyl and isobutyl substitutions also failed to improve the antiviral potency compared to Jun8861. Next, compounds with β-branched substitutions at P1 position were examined. It was found that compounds Jun11762 (cyclohexyl), Jun11808 (cyclopentyl), Jun11718 (isopropyl), and Jun11756 (isobutyl) had comparable enzymatic inhibition and antiviral activity as Jun8861 but had improved cytotoxicity (CC₅₀>150 μM). In comparison, Jun11817 and Jun11861 with the benzyl substitutions were less active. Jun11724 with the tert-butyl substitution was inactive (IC₅₀>20 μM). Similarly, compounds Jun11806 and Jun11805 with α-branched substitutions were also less active compared to Jun11762. Collectively, the structure-activity relationship studies revealed Jun11762 as the most potent lead with improved enzymatic inhibition (IC₅₀=0.09 vs 0.18 μM), antiviral activity (EC₅₀=0.13 vs 0.45 μM), cellular toxicity (CC₅₀>150 vs 83.21 μM) compared to telaprevir.

### Example 5. Broad-Spectrum Antiviral Activity of Lead Compounds

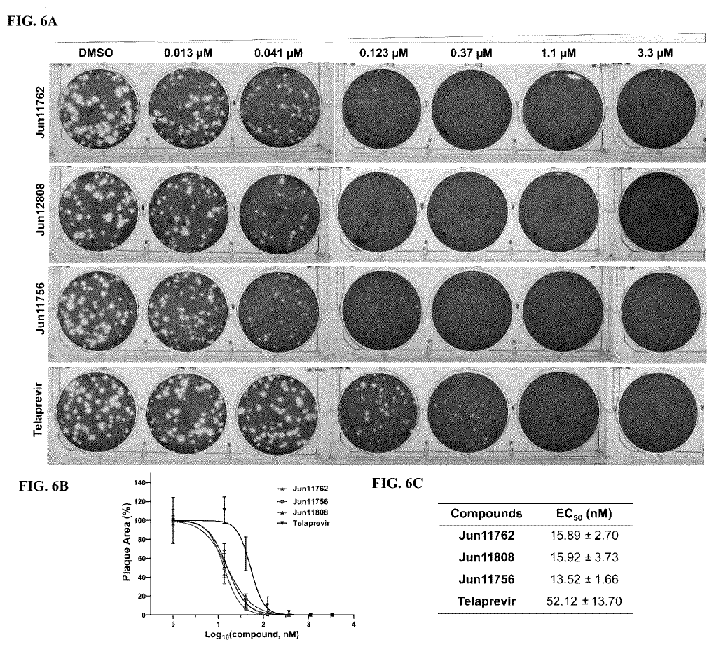

The three most potent hits, Jun11762, Jun12808, and Jun11756, were selected based on the CPE assay results and tested them in the secondary plaque assay. Telaprevir was included as a control. Jun11762 (EC₅₀=15.89 nM), Jun12808 (EC₅₀=15.92 nM), and Jun11756 (EC₅₀=13.52 nM) showed 3 to 4-fold improvement compared to telaprevir (EC₅₀=52.12 nM) (FIG. 6), which is consistent with the CPE assay results.

As the 2A^{pro} is conserved among EV-D68, 2A^{pro} inhibitors are expected to have broad-spectrum antiviral activity. To test this hypothesis, we tested Jun11762 against four additional EV-D68 strains in the B1, B2, and D clades. It was found Jun11762 inhibited all five strains with EC₅₀ values from 43.0 to 268.7 nM, corresponding to a 3.1-4.6-fold improvement compared to telaprevir (Table 1).

TABLE 1
Broad-spectrum antiviral activity of
Jun11762 against five strains of EV-D68.

| | EC₅₀ (nM) | |
|---|---|---|
| EV-D68 viruses | Jun11762 | Telaprevir |
| US/MO/14-18947 | 43.0 ± 3.3 | 199.1 ± 29.1 |
| (clade B1) | | |
| US/MO/14-18949 | 101.0 ± 12.8 | 368.4 ± 34.4 |
| (clade B1) | | |
| US/IL/14-18952 | 268.7 ± 21.9 | 828.4 ± 106.6 |
| (clade B2) | | |
| US/IL/14-18956 | 246.0 ± 34.8 | 836.0 ± 68.5 |
| (clade B2) | | |
| US/KY/14-18953 | 46.7 ± 6.6 | 209.4 ± 37.1 |
| (clade D) | | |

### SUMMARY

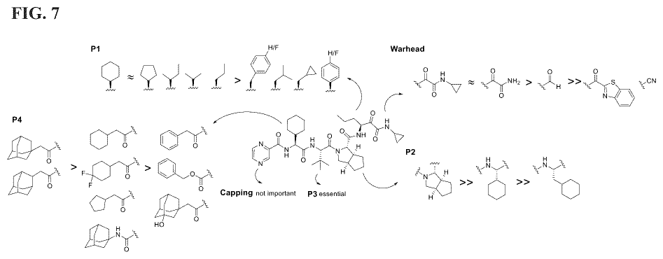

The structure-activity relationship of telaprevir in inhibiting EV-D68 2A^{pro} is summarized in FIG. 7. P1 to P4 substitutions and a reactive warhead are essential for enzymatic inhibition and antiviral activity. The terminal capping group is not essential. For the reactive warhead, α-ketoamide is preferred over aldehyde. Ketobenzothiazole and nitrile are not tolerated, although both were shown to be preferred warheads for SARS-CoV-2 main protease inhibition. For the P1 substitution, cyclohexyl, cyclopentyl, isopropyl, propyl, and isobutyl are preferred over phenyl, cyclopropyl, and benzyl. For the P2 substation, cyclopentylproline is preferred over cyclohexyl and cyclohexylmethyl. The P3 substitution is essential and its deletion leads to a significant decrease of enzymatic inhibition. For the P4 substitution, 1, and 2-adamantylmethyl are preferred over cyclohexylmethyl, cycloheptylmethyl, benzyl, Cbz, and (3-hydroxyl)adamantylmethyl.

### Example 6. Synthesis

The 2A^{pro} inhibitors were assembled using standard solution phase peptide chemistry (Scheme 1). For compounds with different terminal urea or amide capping groups (Scheme 1A), the advanced intermediate 7 was prepared, which subsequently reacted with isocyanate or coupled with carboxylic acid to give the intermediates 8 and 9, respectively. Final step oxidation using Dess-Martin periodinane (DMP) gave the final products Jun1023, Jun7453, Jun8862, Jun8681, Jun8813, Jun971, Jun8814, Jun975, Jun8861, Jun922, and Jun1024.

For compounds with various R₃ substitutions at the P1 position, two synthesis routes were designed (Scheme 1B and C), depending on the availability of starting materials. For amino acids available in the ester form, intermediate 13 was prepared by coupling 12 with the amino ester (Scheme 1B). The ester in intermediate 13 was reduced to hydroxyl by NaBH₄, which was then oxidized to aldehyde 14 by DMP. Next, Passerini reaction involving aldehyde 14, cyclopropyl isocyanide, and acetic acid gave intermediate 15, which was subjected to ester hydrolysis and DMP oxidation to give final products Jun11671, Jun11862, Jun11672, Jun11761, Jun11817, Jun11861, Jun11762, Jun11808, Jun11718, Jun11756, Jun11724, Jun11806, and Jun11805. The amino acids available in the Boc-protected form were first converted to Weinreb amides, which were reduced to aldehydes 17 by LiAlH₄ (Scheme 1C). Next, Passerini reaction by reacting aldehyde 17 with cyclopropyl isocyanide and acetic acid gave the ester intermediate 18, which was converted to the amino alcohol 20 by ester hydrolysis and Boc deprotection. Coupling of acid 12 with the amino alcohol 20 gave intermediate 21, which was oxidized by DMP to give the final products Jun11385, Jun11763, Jun11818, and Jun11389.

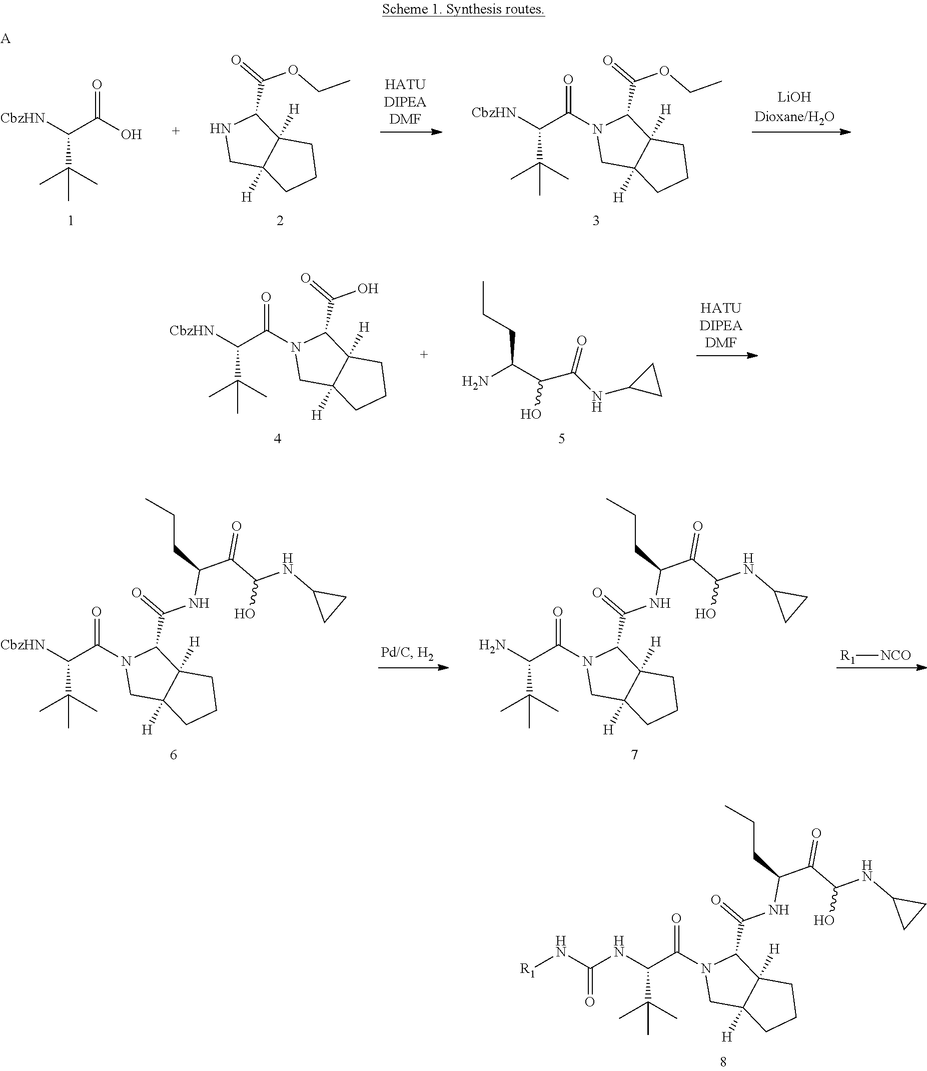

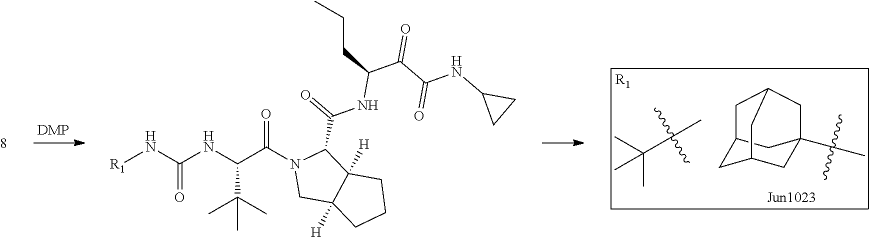

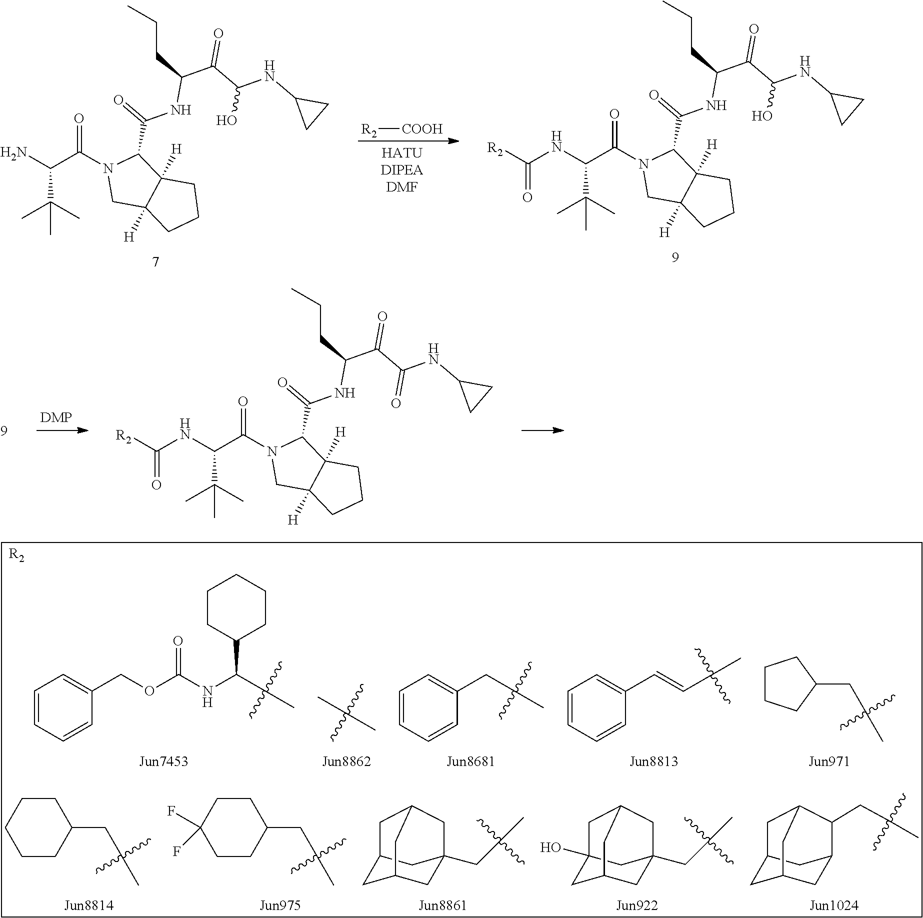

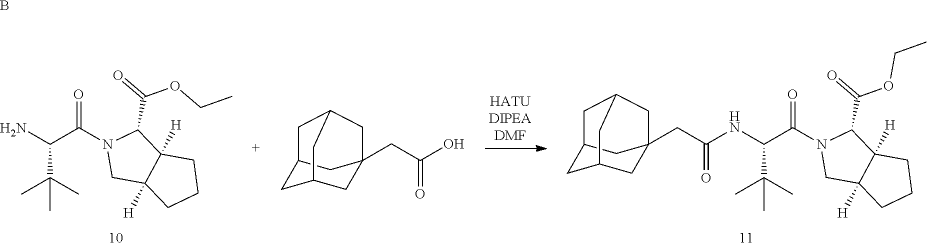

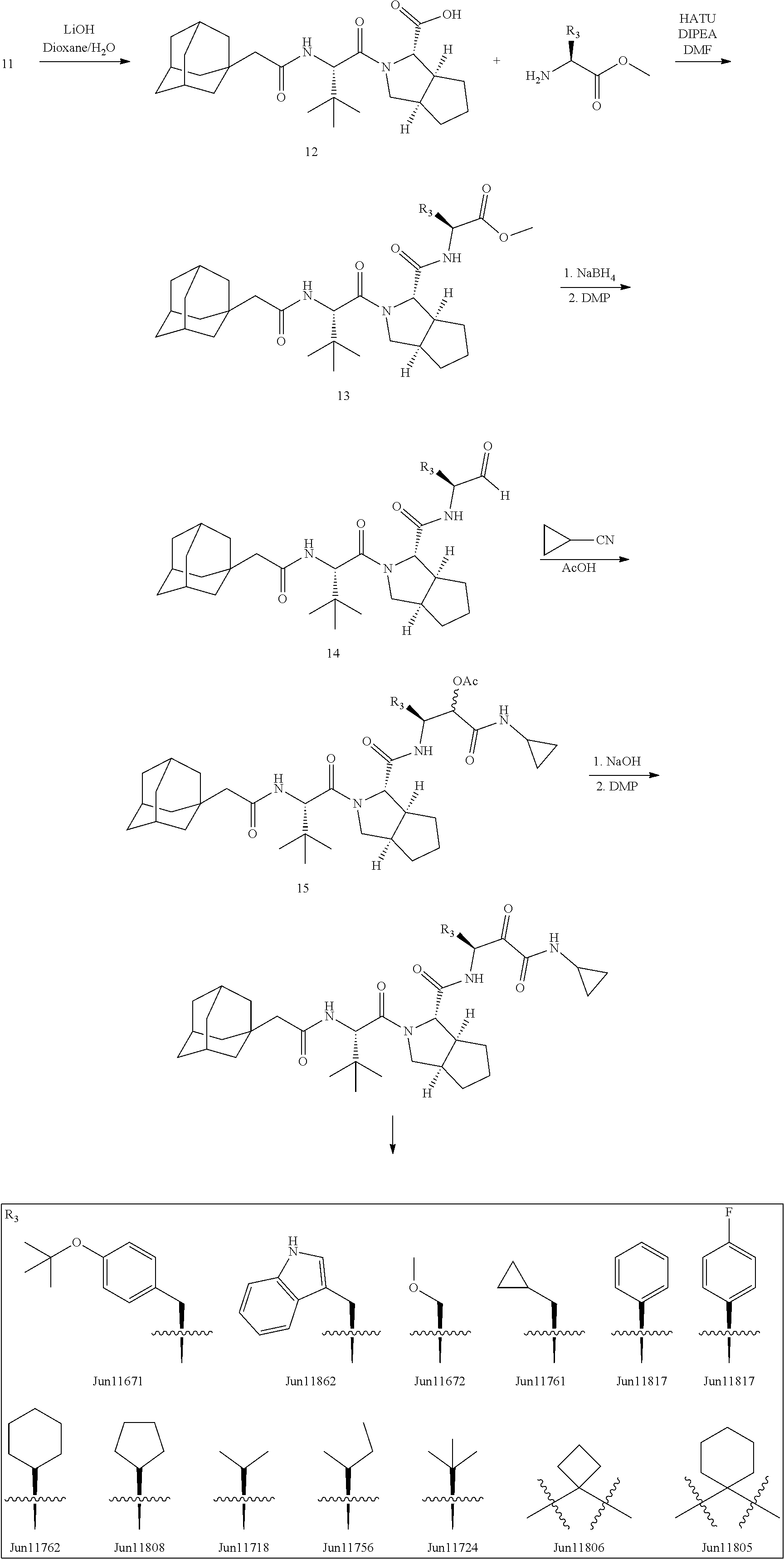

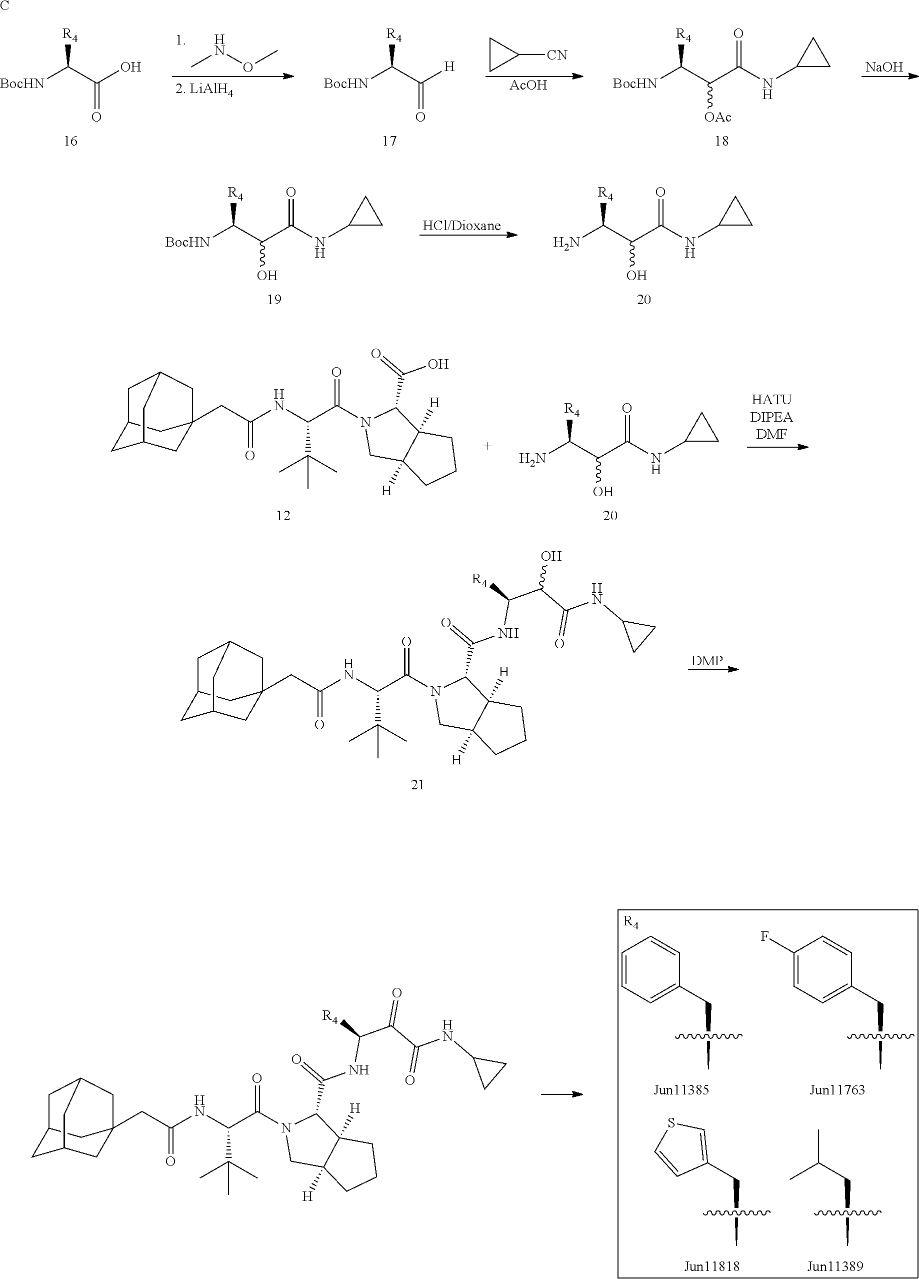

### Example 7. Biology

### Materials and Methods

Cell lines and viruses. Human rhabdomyosarcoma (R.D., ATCC, CCL-136) cells were maintained at 37° C. in a 5% CO₂ atmosphere and cultured in Dulbecco's modified Eagle's medium (DMEM) supplemented with 10% fetal bovine serum (FBS) and 1% penicillin-streptomycin antibiotics. The following reagents were obtained through BEI Resources, NIAID, NIH: Human Enterovirus D68, US/MO/14-18949, NR-49130; Enterovirus D68, US/MO/14-18947, NR-49129; Enterovirus D68, US/IL/14-18952, NR-49131; Enterovirus D68, US/KY/14-18953, NR-49132; Enterovirus D68, US/IL/14-18956, NR-49133.

Antiviral assays. Cells for antiviral assays were seeded and grown overnight at 37° C. in a 5% CO₂ atmosphere for ˜90% confluency the next day. For all infections, cells were washed with PBS containing magnesium and calcium and infected with virus diluted in DMEM with 2% FBS and 30 mM MgCl₂. Viruses were incubated for at least 1 h at 33° C. in a 5% CO₂ atmosphere, followed by the addition of compound as well as 1% penicillin-streptomycin. For CC₅₀ measurements, the experiment was performed similarly but excluded viral infection. For cytopathic effect (CPE) assays, cells were stained with 66 g/mL neutral red for 2 h and neutral red uptake was measured at absorbance 540 nm using a Multiskan FC Microplate Photometer (ThermoFisher Scientific). The EC₅₀ and CC₅₀ values were calculated from best-fit dose response curves using GraphPad Prism. For plaque reduction assays, an 1.2% avicel microcrystalline cellulose (FMC BioPolymer, Philadelphia, PA) overlay was used and cells were stained after 3 days as previously described²⁶.

Protein expression and purification. EV-D68 2A^{pro} gene from strain US/KY/14-18953 was ordered from Genscript (Piscataway, NJ) in pET28b(+) vector with E. coli codon optimization. 2A^{pro} gene was subcloned into pE-SUMOstar vector according to the manufacturers protocol. Plasmid encoding WT-2A^{pro} was transformed into BL21(DE3) competent E. coli cells, and a single colony was picked and used to inoculate 10 mL of L.B. grown supplemented with 50 μg/mL kanamycin at 37° C. at 250 rpm. The 10 mL inoculant was added to 1 L of L.B. with 50 μg/mL kanamycin and grown to an OD600 of 0.8-1.0, cooled to 18° C., then induced using 0.5 mM IPTG. Induced cultures were incubated at 18° C. for an additional 24 h, then harvested and resuspended in lysis buffer (25 mM Tris (pH 7.5); 750 mM NaCl, 2 mM DTT with 0.5 mg/mL lysozyme, 0.5 mM PMSF and 0.02 mg/mL DNase I) and lysed with alternating sonication and French press cycles. The cell debris were removed by centrifugation at 12,000×g for 45 min (20% amplitude, 1 s on/off). The supernatant was incubated with Ni-NTA resin for over 2 h at 4° C. on a rotator. The Ni-NTA resin was thoroughly washed with 50 mM imidazole in wash buffer (50 mM Tris (pH 7.5), 150 mM NaCl, and 2 mM DTT); for the SUMO-tagged protein, the protein was directly eluted with 300 mM imidazole; for the nontagged 2A^{pro} Ni-NTA resin was treated with SUMO Protease 1 to remove the SUMO tag, then 2A^{pro} was washed off from the column with 10 mM imidazole in wash buffer. The imidazole was removed via dialysis or 10 K molecular weight cut-off centrifugal concentrator spin column. The purity of the protein was confirmed with SDS-PAGE. The protein concentration was determined by classic BCA assay using bovine serum albumin as a standard. EV-D68 3C^{pro} in pET28b(+) vector was expressed as previously described¹⁰.

Protein crystallization. Crystallization of EV-D68 2A^{pro} protein (10 mg/ml) was carried out using hanging-drop vapor diffusion at 20° C. Crystals of EV-D68 2A^{pro} protein were obtained using a well solution containing 0.05 M sodium cacodylate (pH 6.0), 15% (v/v) 2-propanol, 0.05 M MgCl₂, 0.002 M CaCl₂), and 0.001 M Spermine. The EV-D68 2A^{C107A} (13 mg/ml) protein crystallized under similar conditions to the wild-type protein. Crystals were generated by hanging-drop vapor diffusion using a well solution containing 0.05 M sodium cacodylate (pH 6.5), 10% (v/v) 2-propanol, 0.005 M MgCl₂ and 0.005 M Spermine at 20° C. Crystals were incubated for 10 minutes in a mother liquor solution containing 40% glycerol as a cryoprotectant before flash freezing in liquid nitrogen.

Data Collection and Processing. X-ray diffraction data were collected remotely at the National Synchrotron Light Source II beamline 17-ID-2 using an Eiger 16 M detector. Single crystal diffraction data were processed by X.D.,²⁷ and the space group was determined to belong to P4. Partial twining was detected by Xtriage in Phenix.

Structure Solution and Refinement. The phase of EV-D68 2A^{C107A} was determined using molecular replacement (M.R.) by the program BALBES on the CCP4 online server²⁹ using the structure of human rhinovirus 2 (HRV2) 2A^{pro} (PDB code: 2HRV) as the search model. To achieve a model with better statistics and geometry, a second round of molecular replacement was done using the initial M.R. model (Phaser-MR in Phenix), along with a poly-Alanine model to reduce bias. The model was further refined by multiple rounds of refinement, followed by Rosetta refinement (Phenix) (twin operator applied with default setting) and Buster refinement. ³⁰ Ramachandran and rotamer outliers were manually fixed using KING. The structure of EV-D68 2A^{pro} was solved in the same way as the described above using EV-D68 2A^{C107A} as the search model.

Peptide synthesis. The FRET substrate of EV-D68 2A^{pro}, Dabcyl-KIRIVNT/GPGFGGE-Edans, was synthesized using the Fmoc solid phase synthesis strategy.

Enzymatic assays. The reaction buffer contains 50 mM Tris (pH7.0), 150 mM NaCl, 10% Glycerol, and 2 mM DTT, and the reaction was carried out at 30° C. in Cytation 5 imaging reader (Fisher Scientific) with EX360/40-EM460/40 filter. Reactions were monitored every 90 sec. For telaprevir IC₅₀ measurements, 1.0 μM 2A^{pro} was incubated with varying concentrations of telaprevir at 30° C. for 1 hour in reaction buffer. The reaction was initiated by adding 20 μM of FRET substrate, and the reaction was monitored for 2 hours and initial velocity was calculated for the first 30 minutes via linear regression. The IC₅₀ was calculated by plotting the initial velocity against various concentrations of telaprevir with the dose-response curve in Prism 5.0.

### Example 8. Chemistry

### General Chemical Methods

All chemicals were purchased from commercial vendors and used without further purification unless otherwise noted. ¹H and ¹³C NMR spectra were recorded on a Bruker-400 or -500 NMR spectrometer. Chemical shifts are reported in parts per million referenced with respect to residual solvent (CD₃OD) 3.31 ppm, (DMSO-d₆) 2.50 ppm, and (CDCl₃) 7.26 ppm or from internal standard tetramethylsilane (TMS) 0.00 ppm. The following abbreviations were used in reporting spectra: s, singlet; d, doublet; t, triplet; q, quartet; m, multiplet; dd, doublet of doublets; ddd, doublet of doublet of doublets. All reactions were carried out under N2 atmosphere, unless otherwise stated. HPLC-grade solvents were used for all reactions. Flash column chromatography was performed using silica gel (230-400 mesh, Merck). High resolution mass spectra were obtained using an Orbitrap™ for all the compounds, obtained in an Ion Cyclotron Resonance (ICR) spectrometer. The purity was assessed by using Shimadzu UPLC with Shimdazu C18-AQ column (4.6×150 mm P/N #227-30767-05) at a flow rate of 1 mL/min; λ=254 and 220 nm; mobile phase A, 0.1% trifluoroacetic acid in H₂O, and mobile phase B, 0.1% trifluoroacetic acid in 90% CH₃CN and 10% H₂O. The gradients are 0-2 min 10% B, 2-15 min 10%-100% B, 15-18 min, 100% B, 18.1-20 min 10% B. All compounds submitted for testing were confirmed to be >95.0% purity by HPLC traces. All final products were characterized by proton and carbon NMR, HPLC, and HRMS.

General amide coupling procedure: Corresponding acid (1 eq.), HATU (1.2 eq.) were dissolved in DMF and stirred for 30-60 mins. Then corresponding amine (1-1.2 eq.), DIEA (4 eq.) were added. The reaction mixture was stirred at ambient temperature for 20 hours. Then E.A. and water were added. The organic phase was washed by water, 1 M HCl (aq.), saturated NaHCO₃(aq.), brine, dried by Na₂SO₄, filtered, removed the solvent to give the crude product which can be purified with silica gel chromatography.

General ester hydrolysis procedure: Corresponding ester was dissolved in THF/MeOH/H₂O(1:1:1). LiOH (2 eq.) was added. After 12 hours stirring at ambient temperature, the solvent was partly removed in vacuo and 1N HCl (aq.) was added till the pH=2. The aqueous phase was extracted by DCM 3 times, dried by dried by Na₂SO₄. The solvent was removed in vacuo to give the crude product without purification.

General ester reduction procedure: Corresponding ester was dissolved in MeOH. NaBH₄ (10 eq.) was added slowly at 0° C. After stirring at ambient temperature for 1 hour, the solvent was partly removed in vacuo and 1N HCl (aq.) was added. After 30 minutes stirring, E.A. was added. The organic phase was separated and dried with NaSO4. The solvent was removed in vacuo and purified with silica gel chromatography or without further purification.

General Passerini reaction procedure: Corresponding aldehyde was dissolved in E.A. Corresponding isocyanide (1.2 eq.), and acetic acid (1.2 eq.) were added at 0° C. The reaction mixture was stirred at ambient temperature for 12 hours till the starting martial disappeared. After removed the solvent in vacuo, the crude product was purified with silica gel chromatography or without further purification.

General Dess-Martin periodinane oxidation procedure: Corresponding alcohol was dissolved in anhydrous DCM and Dess-Martin periodinane (1.5 eq.) was added at 0° C. The reaction mixture was stirred at ambient temperature for 1 hour then the solvent was removed in vacuo. 10% Na₂S₂O₃ (aq.) was added and stirred for 30 min then E.A. was added. The organic phase was washed by 10% Na₂S2O₃ (aq.), saturated NaHCO₃(aq.), brine, dried by Na₂SO₄. The solvent was removed in vacuo and purified with silica gel chromatography.

General Cbz deprotection procedure: Corresponding material (1 eq.), wet palladium/carbon (5% based on palladium) were dissolved in MeOH. The reaction mixture was stirred at ambient temperature under H2 (1 atm, balloon) for 12 hours. The solid was filtered and the solvent was removed in vacuo to give the product without further purification.

General Boc deprotection procedure: Corresponding material (1 eq.) was added 4 M HCl in dioxane (1 mL/mmol) at 0° C. under N2. The reaction mixture was stirred at ambient temperature for 1 hour. The solvent was removed in vacuo to give the product without further purification.

### Example 9. Preparation of Jun9475 and Jun1023

ethyl (1S,3aR,6aS)-2-((S)-2-(((benzyloxy)carbonyl)amino)-3,3-dimethylbutanoyl)octahydrocyclopenta[c]pyrrole-1-carboxylate (3). Compound 3 was synthesized from starting materials 1 and 2 using the general amide coupling procedure. Yield: 68%. ¹H NMR (400 MHz, CDCl₃) δ 7.41-7.21 (m, 5H), 5.46 (d, J=9.8 Hz, 1H), 5.11-5.00 (m, 2H), 4.38-4.29 (m, 2H), 4.16 (m, 2H), 3.85 (m, 1H), 3.69 (m, 1H), 2.77-2.58 (m, 2H), 1.89 (m, 2H), 1.74 (m, 3H), 1.45 (m, 1H), 1.24 (t, J=7.1 Hz, 3H), 1.02 (s, 9H).

Compound 4 was synthesized from compound 3 by following the general ester hydrolysis procedure and was used for the next step without further purification.

Compound 6 was synthesized from starting materials 4 and 5 following the general amide coupling procedure and used for the next step without further purification.

Compound 7 was synthesized from compound 6 following the general Cbz deprotection procedure and used for the next step without further purification.

Jun9475 and Jun1023 were synthesized from compound 7 through a two-step procedure. In the first step, compound 7 (1 eq), triethylamine (1.2 eq), and isocyanate (1 eq) were added to anhydrous DCM at 0° C. The solution was warmed to room temperature and stirred overnight. The solution was extracted with 1N HCl and brine to give intermediate 8. In the second step, compound 8 was oxidized by following the general DMP oxidation procedure to give the final products Jun9475 and Jun1023.

(1S,3aR,6aS)-2-((S)-2-(3-(tert-butyl)ureido)-3,3-dimethylbutanoyl)-N—((S)-1-(cyclopropylamino)-1,2-dioxohexan-3-yl)octahydrocyclopenta[c]pyrrole-1-carboxamide (Jun9475). White solid. Yield: 56%. ¹H NMR (400 MHz, CDCl₃) δ 7.42 (d, J=6.8 Hz, 1H), 6.94 (d, J=3.6 Hz, 1H), 5.34-5.21 (m, 1H), 4.98 (d, J=9.6 Hz, 1H), 4.55-4.43 (m, 3H), 3.85 (dd, J=10.8, 4.0 Hz, 1H), 3.74 (dd, J=10.4, 8.0 Hz, 1H), 3.51 (d, J=3.2 Hz, 1H), 2.98-2.89 (m, 1H), 2.85-2.76 (m, 2H), 1.96-1.81 (m, 3H), 1.65-1.56 (m, 3H), 1.48-1.36 (m, 3H), 1.33 (s, 9H), 1.01 (s, 9H), 0.93 (t, J=7.2 Hz, 3H), 0.89-0.84 (m, 2H), 0.66-0.58 (m, 2H). ¹³C NMR (100 MHz, CDCl₃) δ 196.6, 172.9, 171.3, 160.5, 157.0, 66.0, 57.3, 54.2, 54.2, 50.4, 45.0, 43.2, 35.2, 33.5, 32.1, 31.6, 29.4, 26.5, 25.4, 22.4, 19.1, 13.6, 6.5, 6.5. C₂₈H₄₇N₅O₅ ESI-MS: m/z (M+H⁺): 534.7 (calculated), 534.4 (found).

(1S)-2-((S)-2-(3-((3R,5R,7R)-adamantan-1-yl)ureido)-3,3-dimethylbutanoyl)-N—((S)-1-(cyclopropylamino)-1,2-dioxohexan-3-yl)octahydrocyclopenta[c]pyrrole-1-carboxamide (Jun 1023). White solid. Yield: 71%. ¹H NMR (400 MHz, CDCl₃) δ 7.82 (d, J=7.1 Hz, 1H), 7.07 (d, J=4.1 Hz, 1H), 5.50 (d, J=9.8 Hz, 1H), 5.20-5.15 (m, 1H), 4.86 (s, 1H), 4.55 (d, J=9.7 Hz, 1H), 4.44 (d, J=3.2 Hz, 1H), 3.83-3.77 (m, 2H), 2.84-2.71 (m, 4H), 2.04 (s, 4H), 1.93 (s, 6H), 1.88-1.84 (m, 3H), 1.65 (s, 7H), 1.39 (d, J=7.3 Hz, 2H), 0.97 (s, 10H), 0.90 (t, J=7.3 Hz, 4H), 0.84 (d, J=7.4 Hz, 2H), 0.63-0.60 (m, 2H). ¹³C NMR (101 MHz, CDCl₃) δ 197.25, 172.71, 171.85, 160.84, 156.71, 65.99, 56.99, 54.49, 54.27, 50.67, 46.16, 43.23, 42.43, 36.43, 35.55, 33.29, 32.07, 31.52, 29.51, 26.51, 25.43, 22.50, 19.09, 13.54, 6.46, 6.42. C₃₄H₅₃N₅O₅, HRMS calculated for m/z [M+H]⁺: 612.4125 (calculated), 612.4128 (found).

### Example 10. Preparation of Jun7453, Jun8862, Jun8681, Jun8813, Jun971, Jun8814, Jun975, Jun8861, Jun922, and Jun1024

Jun7453, Jun8862, Jun8681, Jun8813, Jun971, Jun8814, Jun975, Jun8861, Jun922, and Jun1024 were synthesized from compound 7 through a two-step procedure. In the first step, compound 9 was synthesized from compound 7 by following the general amide coupling reaction. In the second step, compound 9 was oxidized by following the general DMP oxidation procedure to give the final product.

Benzyl ((S)-1-cyclohexyl-2-(((S)-1-((1S,3aR,6aS)-1-(((S)-1-(cyclopropylamino)-1,2-dioxohexan-3-yl)carbamoyl)hexahydrocyclopenta[c]pyrrol-2(1H)-yl)-3,3-dimethyl-1-oxobutan-2-yl)amino)-2-oxoethyl)carbamate (Jun7453). White solid. Yield: 51%. ¹H NMR (400 MHz, CDCl₃) δ 7.90 (dd, J=21.4, 6.7 Hz, 2H), 7.62 (d, J=9.5 Hz, 1H), 7.28 (m, 5H), 5.60 (d, J=9.3 Hz, 1H), 5.47 (d, J=9.6 Hz, 1H), 5.12-5.03 (m, 2H), 4.77 (d, J=9.6 Hz, 1H), 4.61 (s, 1H), 4.32 (m, 1H), 3.93 (m, 1H), 3.57 (m, 1H), 2.95-2.70 (m, 3H), 2.59 (m, 1H), 1.75 (m, 6H), 1.55 (t, J=12.2 Hz, 4H), 1.41 (m, 6H), 1.23-0.98 (m, 5H), 0.95 (s, 1H), 0.87 (s, 9H), 0.74 (m, 2H), 0.69-0.61 (m, 2H). ¹³C NMR (101 MHz, CDCl₃) δ 198.52, 171.36, 171.33, 170.21, 160.29, 156.22, 136.28, 128.45, 128.05, 127.66, 66.78, 65.72, 59.67, 56.39, 55.14, 53.17, 46.18, 42.84, 40.43, 36.75, 33.44, 32.82, 32.53, 29.36, 28.78, 26.36, 25.99, 25.78, 22.72, 19.17, 13.19, 6.07. C₃₉H₅₇N₅O₇, HRMS calculated for m/z [M+H]⁺: 708.6336 (calculated), 708.5358 (found).

(1S,3aR,6aS)-2-((S)-2-acetamido-3,3-dimethylbutanoyl)-N—((S)-1-(cyclopropylamino)-1,2-dioxohexan-3-yl)octahydrocyclopenta[c]pyrrole-1-carboxamide (Jun8862). White solid. Yield: 56%. ¹H NMR (400 MHz, DMSO-d₆) δ 9.23 (d, J=8.2 Hz, 1H), 8.74-8.65 (m, 1H), 8.30-8.18 (m, 1H), 5.03-4.92 (m, 1H), 4.50 (d, J=8.3 Hz, 1H), 4.28 (dd, J=6.7, 3.8 Hz, 1H), 3.82-3.67 (m, 1H), 3.65-3.55 (m, 1H), 2.80-2.71 (m, 1H), 2.68-2.61 (m, 1H), 2.51-2.46 (m, 1H), 1.87-1.50 (m, 7H), 1.50-1.33 (m, 6H), 1.03-0.97 (m, 6H), 0.97-0.83 (m, 7H), 0.72-0.61 (m, 2H), 0.61-0.53 (m, 2H). ¹³C NMR (101 MHz, DMSO-d₆) δ 197.48, 172.17, 167.98, 162.57, 162.47, 65.53, 58.56, 54.46, 53.83, 47.77, 43.04, 35.28, 32.12, 31.75, 28.55, 26.76, 26.65, 25.12, 22.92, 19.17, 13.93, 13.89, 5.87, 5.82. C₂₅H₄₀N₄O₅, HRMS calculated for m/z [M+H]⁺: 477.3077 (calculated), 477.3188 (found).

(1S,3aR,6aS)—N—((S)-1-(cyclopropylamino)-1,2-dioxohexan-3-yl)-2-((S)-3,3-dimethyl-2-(2-phenylacetamido)butanoyl)octahydrocyclopenta[c]pyrrole-1-carboxamide (Jun8681). White solid. Yield: 72%. ¹H NMR (400 MHz, CDCl₃) δ 7.42-7.25 (m, 5H), 7.13 (d, J=7.1 Hz, 1H), 6.93 (d, J=3.9 Hz, 1H), 6.15 (d, J=9.3 Hz, 1H), 5.31-5.21 (m, 1H), 4.64 (d, J=9.3 Hz, 1H), 4.44 (d, J=2.7 Hz, 1H), 3.75 (d, J=6.1 Hz, 2H), 2.98-2.88 (m, 1H), 2.87-2.74 (m, 2H), 1.97-1.81 (m, 3H), 1.76-1.66 (m, 1H), 1.65-1.52 (m, 2H), 1.55-1.32 (m, 4H), 1.00-0.81 (m, 15H), 0.67-0.57 (m, 2H). ¹³C NMR (101 MHz, CDCl₃) δ 196.22, 170.98, 170.96, 170.62, 160.38, 134.80, 129.21, 128.97, 127.34, 77.34, 77.02, 76.70, 65.99, 56.67, 54.37, 54.09, 45.05, 43.71, 43.13, 35.52, 33.50, 32.14, 31.80, 26.26, 25.54, 22.42, 19.09, 13.58, 6.50, 6.41. C₃₁H₄₄N₄O₅, HRMS calculated for m/z [M+H]⁺: 553.3390 (calculated), 553.4110 (found).

(1S,3aR,6aS)-2-((S)-2-cinnamamido-3,3-dimethylbutanoyl)-N—((S)-1-(cyclopropylamino)-1,2-dioxohexan-3-yl)octahydrocyclopenta[c]pyrrole-1-carboxamide (Jun8813). White solid. Yield: 68%. ¹H NMR (400 MHz, DMSO-d₆) δ 8.71 (d, J=5.2 Hz, 1H), 8.24 (d, J=6.9 Hz, 1H), 8.06 (d, J=8.7 Hz, 1H), 7.58-7.51 (m, 2H), 7.45-7.36 (m, 3H), 7.36-7.29 (m, 1H), 7.01-6.90 (m, 1H), 5.02-4.92 (m, 1H), 4.56 (dd, J=9.2, 3.5 Hz, 1H), 4.25 (d, J=3.9 Hz, 1H), 3.83-3.74 (m, 1H), 3.74-3.67 (m, 1H), 2.80-2.55 (m, 2H), 1.87-1.29 (m, 11H), 1.03-0.95 (m, 9H), 0.90-0.84 (m, 3H), 0.70-0.48 (m, 4H). ¹³C NMR (101 MHz, DMSO-d₆) δ 197.52, 172.34, 169.75, 165.44, 162.57, 139.51, 135.46, 129.88, 129.37, 127.95, 122.49, 65.37, 57.37, 54.38, 53.84, 47.76, 43.04, 40.55, 40.34, 40.13, 39.92, 39.71, 39.50, 39.30, 36.36, 35.13, 32.15, 32.11, 31.83, 26.91, 25.19, 22.94, 19.17, 13.93, 5.89, 5.84. C₃₂H₄₄N₄O₅, HRMS calculated for m/z [M+H]⁺: 565.3390 (calculated), 565.4113 (found).

(1S,3aR,6aS)-2-((S)-2-(2-cyclopentylacetamido)-3,3-dimethylbutanoyl)-N—((S)-1-(cyclopropylamino)-1,2-dioxohexan-3-yl)octahydrocyclopenta[c]pyrrole-1-carboxamide (Jun971). White solid. Yield: 61%. ¹H NMR (500 MHz, DMSO-d₆) δ 8.70 (d, J=5.2 Hz, 1H), 8.21 (d, J=7.0 Hz, 1H), 7.75 (d, J=9.1 Hz, 1H), 5.00-4.92 (m, 1H), 4.47 (d, J=9.1 Hz, 1H), 4.25 (d, J=3.7 Hz, 1H), 3.77-3.69 (m, 1H), 3.70-3.63 (m, 1H), 2.79-2.68 (m, 1H), 2.67-2.57 (m, 1H), 2.23-2.02 (m, 3H), 1.85-1.30 (m, 16H), 1.19-1.06 (m, 2H), 0.91 (d, J=19.8 Hz, 10H), 0.72-0.58 (m, 2H), 0.61-0.52 (m, 2H). ¹³C NMR (126 MHz, DMSO-d₆) δ 197.50, 172.50, 172.35, 170.00, 162.58, 65.25, 56.73, 55.36, 54.33, 53.85, 47.69, 42.91, 41.31, 40.48, 37.55, 34.86, 32.27, 32.20, 32.18, 32.16, 31.92, 26.91, 25.11, 24.98, 24.97, 22.93, 19.18, 13.94, 5.88, 5.82. C₃₀H₄₈N₄O₅, HRMS calculated for m/z [M+H]⁺: 545.3703 (calculated), 545.4003 (found).

(1S,3aR,6aS)-2-((S)-2-(2-cyclohexylacetamido)-3,3-dimethylbutanoyl)-N—((S)-1-(cyclopropylamino)-1,2-dioxohexan-3-yl)octahydrocyclopenta[c]pyrrole-1-carboxamide (Jun8814). White solid. Yield: 76%. ¹H NMR (400 MHz, DMSO-d₆) δ 8.71 (d, J=5.2 Hz, 1H), 8.22 (d, J=6.9 Hz, 1H), 7.76 (d, J=9.0 Hz, 1H), 5.01-4.91 (m, 1H), 4.48 (d, J=9.1 Hz, 1H), 4.25 (d, J=3.6 Hz, 1H), 3.79-3.64 (m, 2H), 2.81-2.69 (m, 1H), 2.69-2.53 (m, 1H), 2.13-2.02 (m, 2H), 1.81-1.34 (m, 15H), 1.19-1.10 (m, 2H), 0.96-0.87 (m, 14H), 0.71-0.60 (m, 2H), 0.63-0.52 (m, 2H). ¹³C NMR (101 MHz, DMSO-d₆) δ 197.46, 172.33, 172.15, 169.99, 162.55, 65.23, 56.72, 55.34, 54.34, 53.84, 47.66, 43.04, 42.86, 35.58, 34.77, 32.85, 32.83, 32.30, 32.13, 31.95, 26.89, 26.35, 26.13, 26.08, 25.09, 22.93, 19.18, 13.93, 5.87, 5.82. C₃₁H₅₀N₄O₅, HRMS calculated for m/z [M+H]⁺: 559.3859 (calculated), 559.4583 (found).

(1S,3aR,6aS)—N—((S)-1-(cyclopropylamino)-1,2-dioxohexan-3-yl)-2-((S)-2-(2-(4,4-difluorocyclohexyl)acetamido)-3,3-dimethylbutanoyl)octahydrocyclopenta[c]pyrrole-1-carboxamide (Jun975). White solid. Yield: 71%. ¹H NMR (500 MHz, DMSO-d₆) δ 8.70 (d, J=5.2 Hz, 1H), 8.21 (d, J=6.9 Hz, 1H), 7.83 (d, J=8.9 Hz, 1H), 5.00-4.92 (m, 1H), 4.46 (d, J=9.0 Hz, 1H), 4.25 (d, J=3.7 Hz, 1H), 3.77-3.70 (m, 1H), 3.70-3.64 (m, 1H), 2.79-2.70 (m, 1H), 2.67-2.57 (m, 1H), 2.25-2.05 (m, 2H), 1.99-1.91 (m, 2H), 1.83-1.35 (m, 6H), 1.24-1.15 (m, 2H), 0.96-0.87 (m, 12H), 0.70-0.61 (m, 2H), 0.64-0.53 (m, 2H). ¹³C NMR (126 MHz, DMSO-d₆) δ 197.51, 172.33, 171.78, 169.92, 162.57, 65.26, 56.88, 55.36, 54.37, 53.85, 47.67, 42.90, 34.78, 32.96, 32.89, 32.28, 32.17, 31.95, 28.58, 28.51, 26.90, 25.10, 22.93, 19.18, 13.93, 5.88, 5.82. C₃₁H48F2N₄O₅, HRMS calculated for m/z [M+H]⁺: 595.3671 (calculated), 595.4499 (found).

(1S,3aR,6aS)—N—((S)-1-(cyclopropylamino)-1,2-dioxohexan-3-yl)-2-((S)-3,3-dimethyl-2-(2-phenylacetamido)butanoyl)octahydrocyclopenta[c]pyrrole-1-carboxamide (Jun8861). White solid. Yield: 63%. ¹H NMR (400 MHz, DMSO-d₆) δ 8.70 (d, J=5.2 Hz, 1H), 8.21 (d, J=6.8 Hz, 1H), 7.60 (d, J=9.2 Hz, 1H), 5.01-4.91 (m, 1H), 4.51 (d, J=9.2 Hz, 1H), 4.26 (d, J=3.5 Hz, 1H), 3.77-3.69 (m, 2H), 2.81-2.69 (m, 1H), 2.68-2.58 (m, 2H), 2.07-1.99 (m, 1H), 1.94-1.86 (m, 4H), 1.84-1.70 (m, 1H), 1.69-1.28 (m, 21H), 1.04-0.82 (m, 12H), 0.72-0.62 (m, 2H), 0.61-0.55 (m, 2H). ¹³C NMR (101 MHz, DMSO-d₆) δ 197.43, 172.33, 170.78, 170.08, 162.55, 65.17, 56.44, 54.46, 53.86, 49.49, 47.65, 42.80, 42.48, 42.33, 36.92, 34.70, 32.98, 32.46, 32.13, 28.58, 26.91, 25.16, 22.93, 19.19, 13.93, 5.87, 5.82. C₃₅H₅₄N₄O₅, HRMS calculated for m/z [M+H]⁺: 611.4276 (calculated), 611.5035 (found).

(1S,3aR,6aS)—N—((S)-1-(cyclopropylamino)-1,2-dioxohexan-3-yl)-2-((S)-2-(2-((1r,3R,5R,7S)-3-hydroxyadamantan-1-yl)acetamido)-3,3-dimethylbutanoyl)octa-hydrocyclopenta[c]pyrrole-1-carboxamide (Jun922). White solid. Yield: 57%. ¹H NMR (400 MHz, DMSO-d₆) δ 8.70 (d, J=5.2 Hz, 1H), 8.21 (d, J=6.9 Hz, 1H), 7.63 (d, J=9.2 Hz, 1H), 5.01-4.91 (m, 1H), 4.53-4.43 (m, 1H), 4.31 (s, 1H), 4.26 (d, J=3.6 Hz, 1H), 3.79-3.67 (m, 2H), 2.81-2.69 (m, 1H), 2.66-2.57 (m, 1H), 2.11-1.92 (m, 4H), 1.87-1.24 (m, 22H), 1.04-0.81 (m, 12H), 0.71-0.60 (m, 2H), 0.63-0.54 (m, 2H). ¹³C NMR (101 MHz, DMSO-d₆) δ 197.47, 172.35, 170.73, 170.06, 162.57, 67.17, 65.19, 56.52, 55.35, 54.41, 53.86, 50.87, 48.67, 47.66, 45.07, 45.01, 42.84, 41.35, 41.13, 36.13, 35.75, 34.71, 32.40, 32.14, 32.09, 30.57, 26.94, 25.19, 22.93, 19.19, 13.94, 5.87, 5.82. C₃₅H₅₄N₄O₆, HRMS calculated for m/z [M+H]⁺: 627.4121 (calculated), 627.4238 (found).

(1S)-2-((S)-2-(2-((1S,3S,5S,7S)-adamantan-2-yl)acetamido)-3,3-dimethylbutanoyl)-N—((S)-1-(cyclopropylamino)-1,2-dioxohexan-3-yl)octahydrocyclopenta[c]pyrrole-1-carboxamide (Jun 1024). White solid. Yield: 72%. ¹H NMR (400 MHz, CDCl₃) δ 7.64 (d, J=7.6 Hz, 1H), 7.08 (d, J=4.1 Hz, 1H), 6.65 (d, J=9.6 Hz, 1H), 4.74 (d, J=9.6 Hz, 1H), 4.46 (d, J=2.8 Hz, 1H), 3.86-3.75 (m, 2H), 2.86-2.74 (m, 3H), 2.49-2.42 (m, 1H), 2.31-2.23 (m, 2H), 1.95-1.79 (m, 13H), 1.72 (d, J=4.6 Hz, 4H), 1.66 (dd, J=7.9, 4.9 Hz, 2H), 1.55 (q, J=6.1, 5.3 Hz, 4H), 1.48-1.34 (m, 5H), 0.99 (s, 9H), 0.91 (t, J=7.3 Hz, 4H), 0.84 (d, J=7.2 Hz, 2H), 0.61 (dd, J=8.9, 3.8 Hz, 2H). ¹³C NMR (101 MHz, CDCl₃) δ 196.98, 172.52, 171.43, 171.22, 160.47, 66.10, 56.39, 54.46, 53.88, 53.43, 45.75, 43.11, 41.79, 40.08, 38.82, 38.80, 38.13, 35.33, 33.46, 32.28, 32.00, 31.83, 31.66, 31.47, 31.45, 27.92, 27.79, 26.40, 25.51, 22.42, 19.02, 13.43, 6.36, 6.32. C₃₅H₅₄N₄O₅, HRMS calculated for m/z [M+H]⁺: 612.4172 (calculated), 612.4175 (found).

(S)-3-((S)-2-((S)-2-(2-((3S,5S,7S)-adamantan-1-yl)acetamido)-3,3-dimethylbutanamido)-2-cyclohexylacetamido)-N-cyclopropyl-2-oxohexanamide (Jun10223). White solid. Yield: 81%. ¹H NMR (500 MHz, DMSO-d₆) δ 8.73 (d, J=5.5 Hz, 1H), 8.17 (d, J=6.5 Hz, 1H), 7.85 (d, J=9.0 Hz, 1H), 7.46 (d, J=9.5 Hz, 1H), 5.04-4.96 (m, 1H), 4.32 (d, J=9.5 Hz, 1H), 4.22 (t, J=8.5 Hz, 1H), 2.81-2.71 (m, 1H), 2.04 (d, J=12.55 Hz, 1H), 1.93-1.84 (m, 4H), 1.72-1.26 (m, 24H), 1.19-0.81 (m, 15H), 0.70-0.55 (m, 4H). ¹³C NMR (125 MHz, DMSO-d₆) δ 196.9, 171.2, 170.6, 170.4, 162.3, 59.8, 56.9, 53.6, 49.9, 42.5, 37.0, 34.5, 33.0, 31.7, 29.4, 28.6, 27.2, 26.4, 26.0, 23.0, 19.2, 13.8, 5.9, 5.8. C₃₅H₅₆N₄O₅ HRMS calculated for m/z [M+H]⁺: 613.4329 (calculated), 613.4400 (found).

(S)-3-((S)-2-((S)-2-(2-((3S,5S,7S)-adamantan-1-yl)acetamido)-3,3-dimethylbutanamido)-3-cyclohexylpropanamido)-N-cyclopropyl-2-oxohexanamide (Jun10222). White solid. Yield: 74%. ¹H NMR (500 MHz, DMSO-d₆) δ 8.70 (d, J=5.0 Hz, 1H), 8.07 (d, J=7.0 Hz, 1H), 7.95 (d, J=8.0 Hz, 1H), 7.50 (d, J=9.0 Hz, 1H), 5.01-4.95 (m, 1H), 4.44-4.35 (m, 1H), 4.25 (d, J=9.0 Hz, 1H), 2.80-2.71 (m, 1H), 1.97 (dd, J=23.0, 12.5 Hz, 2H), 1.89 (s, 3H), 1.72-1.07 (m, 27H), 0.96-0.78 (m, 14H), 0.69-0.54 (m, 4H). ¹³C NMR (125 MHz, DMSO-d₆) δ 197.1, 172.5, 170.5, 170.4, 162.4, 60.0, 53.7, 50.1, 49.9, 42.5, 37.0, 34.5, 33.7, 33.6, 32.9, 32.3, 31.9, 28.6, 27.2, 26.6, 26.3, 26.0, 23.0, 19.2, 13.9, 5.9, 5.9. C₃₆H₅₈N₄O₅ HRMS calculated for m/z [M+H]⁺: 627.4485 (calculated), 627.4589 (found).

### Example 11. Preparation of Jun7424

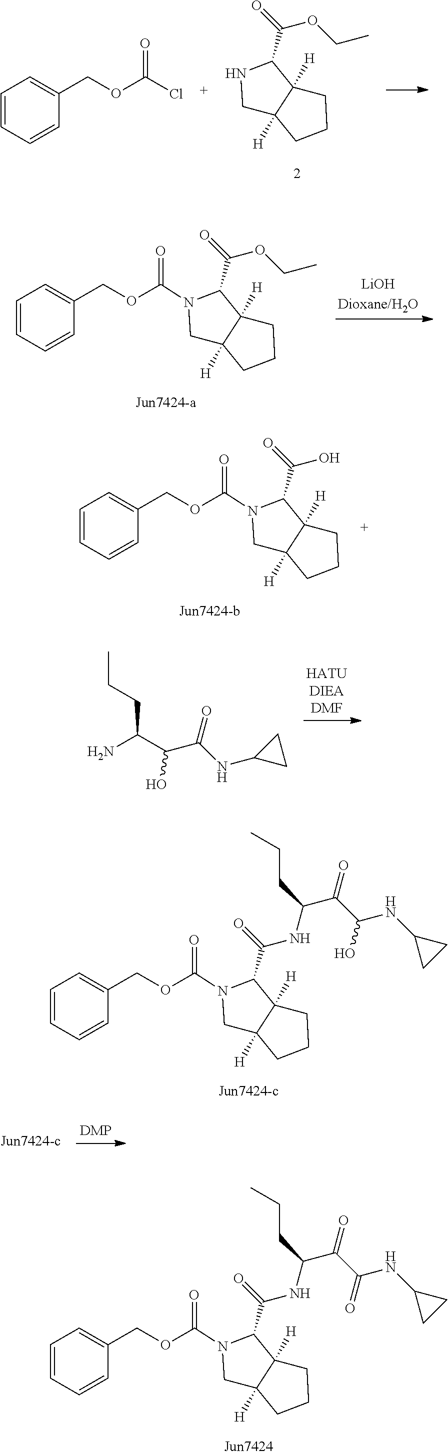

(1S,3aR,6aS)-Octahydrocyclopenta[c]pyrrole-1-carboxylic acid ethyl ester hydrochloride (2) and triethylamine (TEA) were dissolved in dichloromethane (DCM). CbzCl was added dropwise at 0° C. After stirring 12 hours, water was added. The aqueous phase was extracted by DCM 3 times, and dried with Na₂SO₄. The solvent was removed in vacuo to give the crude product, which was purified by silica gel chromatography to give Jun7424-a (90% yield)¹H NMR (400 MHz, CDCl₃) δ 7.46-7.12 (m, 5H), 5.20-4.96 (m, 2H), 4.23-3.95 (m, 3H), 3.75 (m, 1H), 3.35 (m, 1H), 2.73-2.60 (m, 2H), 2.01-1.91 (m, 1H), 1.88-1.68 (m, 2H), 1.62-1.55 (m, 2H), 1.52-1.39 (m, 1H), 1.29-1.08 (m, 3H).

Jun7424-b was synthesized from Jun7424-a following the general ester hydrolysis procedure and used for the next step without further purification.

Jun7424-c was synthesized from Jun7424-b following the general amide coupling procedure and used for the next step without further purification.

Benzyl (1S,3aR,6aS)-1-(((S)-1-(cyclopropylamino)-1,2-dioxohexan-3-yl)carbamoyl)hexahydrocyclopenta[c]pyrrole-2(1H)-carboxylate (Jun7424). Jun7424 was synthesized from Jun7424-c by following the general DMP oxidation procedure. White solid. Yield: 60%. ¹H NMR (400 MHz, DMSO-d₆) δ 8.71 (d, J=5.1 Hz, 1H), 8.33 (d, J=6.7 Hz, 1H), 7.42-7.25 (m, 5H), 5.08-4.96 (m, 2H), 4.91 (m, 1H), 4.12 (dd, J=8.9, 2.8 Hz, 1H), 3.64 (m, 1H), 3.22 (m, 1H), 2.76 (m, 1H), 2.59 (m, 1H), 1.89 (s, 1H), 1.78-1.51 (m, 5H), 1.42 (m, 2H), 1.29 (s, 1H), 0.90 (m, 1H), 0.81 (m, 2H), 0.67 (m, 2H), 0.59 (m, 2H). ¹³C NMR (101 MHz, DMSO-d₆) δ 197.40, 172.85, 162.52, 128.83, 128.64, 128.00, 127.78, 127.39, 66.28, 65.70, 54.03, 53.42, 49.93, 48.67, 42.22, 41.19, 32.72, 32.65, 32.30, 32.05, 31.98, 25.45, 22.93, 19.38, 14.01, 5.87. C₂₅H₃₃N₃O₅ HRMS calculated for m/z [M+H]⁺: 456.2498 (calculated), 456.3018 (found).

### Example 12. Preparation of Jun7426

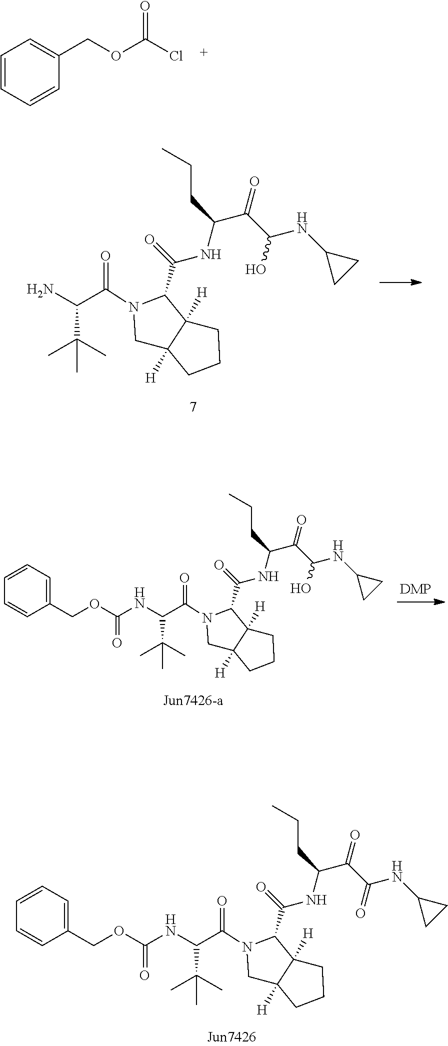

Benzyl ((S)-1-((1S,3aR,6aS)-1-(((S)-1-(cyclopropylamino)-1,2-dioxohexan-3-yl)carbamoyl)hexahydrocyclopenta[c]pyrrol-2(1H)-yl)-3,3-dimethyl-1-oxobutan-2-yl)carbamate (Jun7426). Jun7426 was synthesized by following a similar procedure as Jun7424. White solid. Yield: 40%. ¹H NMR (400 MHz, DMSO-d₆) δ 8.70 (d, J=5.2 Hz, 1H), 8.23 (d, J=6.8 Hz, 1H), 7.38-7.31 (m, 6H), 7.26-7.17 (m, 1H), 5.10-4.96 (m, 3H), 5.00-4.92 (m, 1H), 4.28 (d, J=3.8 Hz, 1H), 4.17 (d, J=8.7 Hz, 1H), 3.79-3.70 (m, 1H), 3.70-3.62 (m, 1H), 2.79-2.72 (m, 1H), 1.86-1.67 (m, 3H), 1.68-1.52 (m, 3H), 1.46-1.32 (m, 3H), 1.00-0.84 (m, 14H), 0.70-0.63 (m, 2H), 0.62-0.56 (m, 2H). ¹³C NMR (101 MHz, DMSO-d₆) δ 197.53, 172.35, 169.97, 162.59, 156.86, 137.57, 128.75, 128.20, 128.08, 65.87, 65.36, 59.51, 54.33, 53.87, 47.65, 43.01, 40.61, 40.40, 40.20, 39.99, 39.78, 39.57, 39.36, 38.71, 34.90, 32.17, 31.86, 26.84, 25.17, 22.93, 19.19, 13.94, 5.88, 5.82. C₃₁H₄₄N₄O₆ HRMS calculated for m/z [M+H]⁺: 569.3339 (calculated), 569.4084 (found).

### Example 13. Preparation of Jun11191

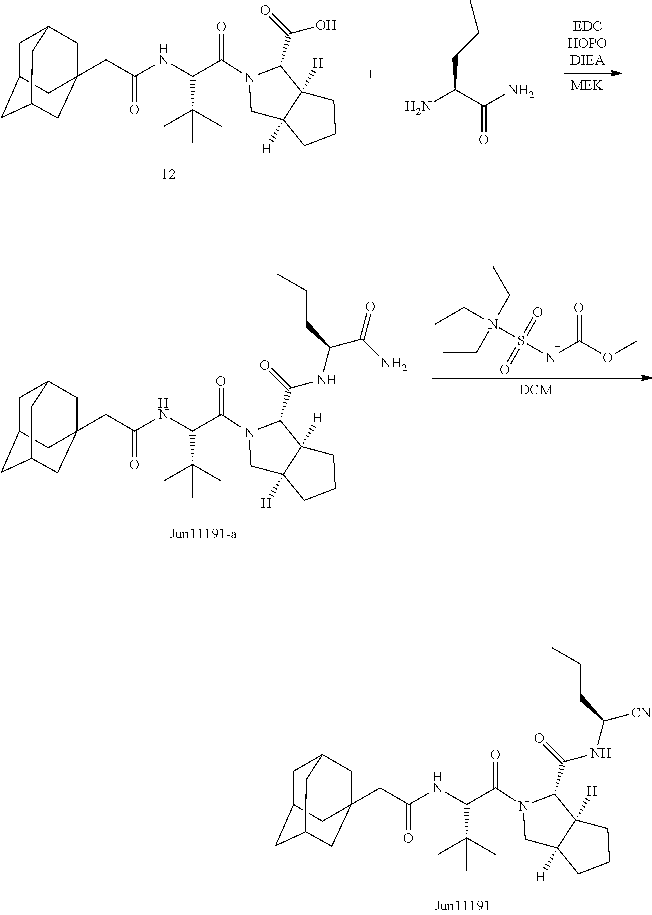

(1S,3aR,6aS)-2-((S)-2-(2-((3S,5S,7S)-adamantan-1-yl) acetamido)-3,3-dimethylbutanoyl)-N—((S)-1-amino-1-oxopentan-2-yl) octahydrocyclopenta[c]pyrrole-1-carboxamide (Jun11191-a). Under nitrogen, 2-Hydroxypyridine 1-oxide (HOPO) (22.8 mg, 0.25 eq) was added to a solution of (1S,3aR,6aS)-2-((S)-2-(2-((3S,5S,7S)-adamantan-1-yl)acetamido)-3,3-dimethylbutanoyl) octahydrocyclopenta[c]pyrrole-1-carboxylic acid (12) (368 mg, 0.82 mmol) and (S)-2-aminopentanamide HCl salt (141 mg, 0.93 mmol) in butan-2-one (MEK) (4 mL), and the mixture was cooled to 0° C. DIPEA (435 μL, 2.5 mmol) was then added, followed by the addition of EDCI (189 mg, 0.98 mmol). The reaction mixture was stirred at 25° C. overnight. The solution was diluted with E.A. The separated organic layer was washed with water and saturated aqueous sodium chloride solution, dried over Na₂SO₄, filtered, and concentrated to afford Jun11191-a as a yellow solid (336.7 mg, 75% yield). ¹H NMR (400 MHz, CDCl₃) δ 7.12 (d, J=8.0 Hz, 1H), 6.53-6.47 (m, 1H), 6.37 (d, J=12.0 Hz, 1H), 5.85-5.80 (m, 1H), 4.71 (d, J=8.0 Hz, 1H), 4.41-4.36 (m, 2H), 3.80 (s, 1H), 2.81 (s, 2H), 1.96-1.95 (m, 6H), 1.70-1.55 (m, 18H), 1.51-1.42 (m, 3H), 1.00 (s, 9H), 0.92-0.87 (m, 3H). C₃₁H₅₀N₄O₄ ESI-MS calculated for m/z [M+H]⁺: 543.8 (calculated), 543.4 (found).

(1S,3aR,6aS)-2-((S)-2-(2-((3S,5S,7S)-adamantan-1-yl) acetamido)-3,3-dimethylbutanoyl)-N—((S)-1-cyanobutyl) octahydro-cyclopenta[c]pyrrole-1-carboxamide (Jun11191). Burgess reagent (320 mg, 1.25 mmol) was added to a solution of the Jun11191-a (271 mg, 0.5 mmol) in dichloromethane (6 mL). The reaction mixture was stirred at 25° C. overnight before being diluted with DCM (10 mL). The reaction mixture was quenched by sat. NaHCO₃ (40 mL), washed by 1 M HCl and brine (40 mL). The separated organic phase was concentrated to get a residue which was purified by flash column chromatography (E.A./hexane=¼-½) to give Jun11191 as a white solid (60 mg, 23% yield). ¹H NMR (400 MHz, CDCl₃) δ 7.65 (d, J=8.0 Hz, 1H), 6.08 (d, J=12.0 Hz, 1H), 4.84 (q, J=8.0 Hz, 1H), 4.67 (d, J=8.0 Hz, 1H), 4.32 (s, 1H), 3.83-3.74 (m, 2H), 2.94-2.85 (m, 8H), 1.97-1.92 (m, 1H), 1.70-1.60 (m, 18H), 1.51-1.46 (m, 3H), 0.99 (s, 9H), 0.94 (d, J=10.0 Hz, 3H). ¹³C NMR (101 MHz, CDCl₃) δ 172.12, 171.09, 170.91, 119.12, 66.41, 56.76, 54.70, 51.64, 45.29, 43.38, 42.81, 40.30, 36.90, 35.36, 35.25, 33.13, 32.41, 31.90, 28.81, 26.70, 25.68, 18.81, 13.33. C₃₁H₄₈N₄O₃ HRMS calculated for m/z [M+H]⁺: 525.3805 (calculated), 525.4193 (found).

### Examples 14-17

Using procedures similar to those described herein, the following compounds were also prepared.

### Example 14

(1S,3aR,6aS)-2-((S)-2-(2-((3S,5S,7S)-adamantan-1-yl)acetamido)-3,3-dimethylbutanoyl)-N—((S)-4-(cyclopropylamino)-3,4-dioxo-1-phenylbutan-2-yl)octahydrocyclopenta[c]pyrrole-1-carboxamide (Jun11385). White solid. Yield: 78%. ¹H NMR (400 MHz, CDCl₃) δ 7.28 (s, 3H), 7.18 (dd, J=14.6, 7.3 Hz, 2H), 6.86 (d, J=7.0 Hz, 1H), 6.02 (d, J=10.1 Hz, 1H), 4.72 (t, J=11.0 Hz, 1H), 4.46-4.31 (m, 1H), 3.78 (dd, J=10.9, 4.9 Hz, 1H), 3.72-3.62 (m, 1H), 3.39-3.25 (m, 1H), 3.20 (dd, J=14.3, 6.9 Hz, 1H), 2.82 (ddt, J=26.3, 14.1, 6.4 Hz, 3H), 1.99 (s, 4H), 1.89 (dt, J=12.9, 6.6 Hz, 2H), 1.73 (d, J=12.1 Hz, 3H), 1.66 (s, 6H), 1.56-1.32 (m, 4H), 1.02 (d, J=8.6 Hz, 3H), 1.01-0.89 (m, 9H), 0.88 (d, J=4.2 Hz, 3H), 0.62 (d, J=8.4 Hz, 2H). ¹³C NMR (101 MHz, DMSO) δ 196.55, 172.43, 170.79, 170.12, 162.37, 137.68, 129.43, 128.72, 126.95, 65.10, 56.40, 55.61, 54.43, 49.47, 47.78, 42.46, 36.91, 34.65, 32.98, 32.56, 32.17, 28.57, 26.94, 25.17, 22.95, 5.87, 5.83. C₃₉H₅₄N₄O₅, HRMS calculated for m/z [M+H]⁺: 659.4172 (calculated), 659.3376 (found).

### Example 15

(1S,3aR,6aS)-2-((S)-2-(2-((3S,5S,7S)-adamantan-1-yl)acetamido)-3,3-dimethylbutanoyl)-N—((S)-4-(cyclopropylamino)-1-(4-fluorophenyl)-3,4-dioxobutan-2-yl)octahydrocyclo-penta[c]pyrrole-1-carboxamide (Jun11763). White solid. Yield: 76%. ¹H NMR (400 MHz, CDCl₃) δ 7.17-7.05 (m, 2H), 7.01-6.88 (m, 4H), 6.08 (d, J=9.6 Hz, 1H), 5.52 (td, J=6.7, 5.3 Hz, 1H), 4.68 (d, J=9.6 Hz, 1H), 4.37 (d, J=2.8 Hz, 1H), 3.80-3.64 (m, 2H), 3.25 (dd, J=14.3, 5.4 Hz, 1H), 3.11 (dd, J=14.3, 6.9 Hz, 1H), 2.78 (dtt, J=16.6, 8.6, 3.6 Hz, 3H), 1.95 (d, J=3.3 Hz, 5H), 1.85 (dd, J=13.4, 7.0 Hz, 2H), 1.74-1.65 (m, 4H), 1.60 (d, J=12.4 Hz, 9H), 1.49-1.42 (m, 1H), 1.37 (dt, J=13.4, 6.6 Hz, 1H), 1.00 (d, J=7.8 Hz, 1H), 0.94 (s, 9H), 0.86 (dt, J=6.8, 2.0 Hz, 2H), 0.60 (dd, J=5.8, 3.1 Hz, 2H). ¹³C NMR (101 MHz, CDCl₃) δ 195.18, 171.54, 171.04, 170.80, 160.35, 131.33, 130.81, 115.62, 66.03, 56.30, 55.35, 54.32, 51.71, 45.67, 42.99, 42.64, 36.73, 35.13, 32.97, 32.23, 31.78, 28.63, 26.46, 25.38, 22.47, 6.52, 6.44. C₃₉H₅₃FN₄O₅, HRMS calculated for m/z [M+H]⁺: 677.4078 (calculated), 677.4344 (found).

### Example 16

(1S,3aR,6aS)-2-((S)-2-(2-((3S,5S,7S)-adamantan-1-yl)acetamido)-3,3-dimethylbutanoyl)-N—((S)-1-(4-fluorophenyl)-3-oxopropan-2-yl)octahydrocyclopenta[c]pyrrole-1-carboxamide (Jun11707). White solid. Yield: 65%. ¹H NMR (400 MHz, CDCl₃) δ 9.60 (d, J=2.8 Hz, 1H), 7.13 (td, J=5.6, 2.8 Hz, 2H), 6.98 (dd, J=6.8, 2.7 Hz, 3H), 4.75-4.66 (m, 2H), 4.33 (q, J=3.5, 2.7 Hz, 1H), 3.81 (dt, J=7.0, 3.0 Hz, 2H), 3.14 (dd, J=6.5, 2.5 Hz, 2H), 2.81 (s, 2H), 2.39-2.31 (m, 1H), 2.05 (d, J=2.7 Hz, 2H), 1.96 (s, 4H), 1.68 (s, 2H), 1.59 (s, 4H), 1.28-1.22 (m, 6H), 1.02 (s, 1H), 0.98 (d, J=2.8 Hz, 9H), 0.94 (d, J=4.0 Hz, 2H). ¹³C NMR (101 MHz, CDCl₃) δ 198.51, 171.66, 171.61, 170.94, 160.71, 139.88-133.10 (m), 130.84, 115.47, 66.20, 59.88, 56.40, 54.40, 51.55, 45.99, 43.11, 42.63, 42.48, 36.73, 35.17, 32.96, 32.24, 31.75, 28.63, 26.49, 25.38. C₃₅H₄₈FN₃O₄, HRMS calculated for m/z [M+H]⁺: 594.3707 (calculated), 594.2794 (found).

### Example 17

(1S,3aR,6aS)-2-((S)-2-(3-((3R,5R,7R)-adamantan-1-yl)ureido)-3,3-dimethylbutanoyl)-N—((S)-4-(cyclopropylamino)-1-(4-fluorophenyl)-3,4-dioxobutan-2-yl)octahydrocyclopenta[c]pyrrole-1-carboxamide (Jun11631). White solid. Yield: 79%. ¹H NMR (400 MHz, CDCl₃) δ 7.20 (d, J=7.0 Hz, 1H), 7.10 (dd, J=8.4, 5.3 Hz, 2H), 6.93 (t, J=8.6 Hz, 3H), 5.49 (q, J=6.4 Hz, 1H), 5.21 (d, J=9.8 Hz, 1H), 4.58-4.44 (m, 2H), 4.40 (d, J=2.6 Hz, 1H), 3.81 (dd, J=10.8, 3.6 Hz, 1H), 3.71 (dd, J=10.7, 7.1 Hz, 1H), 3.47 (s, 1H), 3.23 (dd, J=14.2, 5.8 Hz, 1H), 3.10 (dd, J=14.2, 6.7 Hz, 1H), 2.75 (dtq, J=14.9, 8.1, 3.9 Hz, 3H), 2.06-2.02 (m, 3H), 1.92 (s, 5H), 1.82 (q, J=6.5 Hz, 2H), 1.65 (d, J=6.3 Hz, 5H), 1.51 (ddd, J=11.1, 8.1, 4.7 Hz, 2H), 1.44-1.38 (m, 1H), 0.98 (d, J=8.1 Hz, 1H), 0.93 (s, 9H), 0.85 (d, J=7.4 Hz, 2H), 0.64-0.52 (m, 2H). ¹³C NMR (101 MHz, CDCl₃) δ 195.43, 172.96, 171.43, 160.73, 160.50, 156.70, 131.42, 130.88, 115.56, 66.09, 57.15, 55.43, 54.12, 50.86, 46.12, 43.10, 42.43, 36.41, 35.19, 32.04, 31.43, 29.53, 26.51, 25.25, 22.48, 6.51, 6.44. C₃₈H₅₂FN₅O₅, HRMS calculated for m/z [M+H]⁺: 678.4031 (calculated), 678.3202 (found).

### Example 18. Synthesis of Jun11705

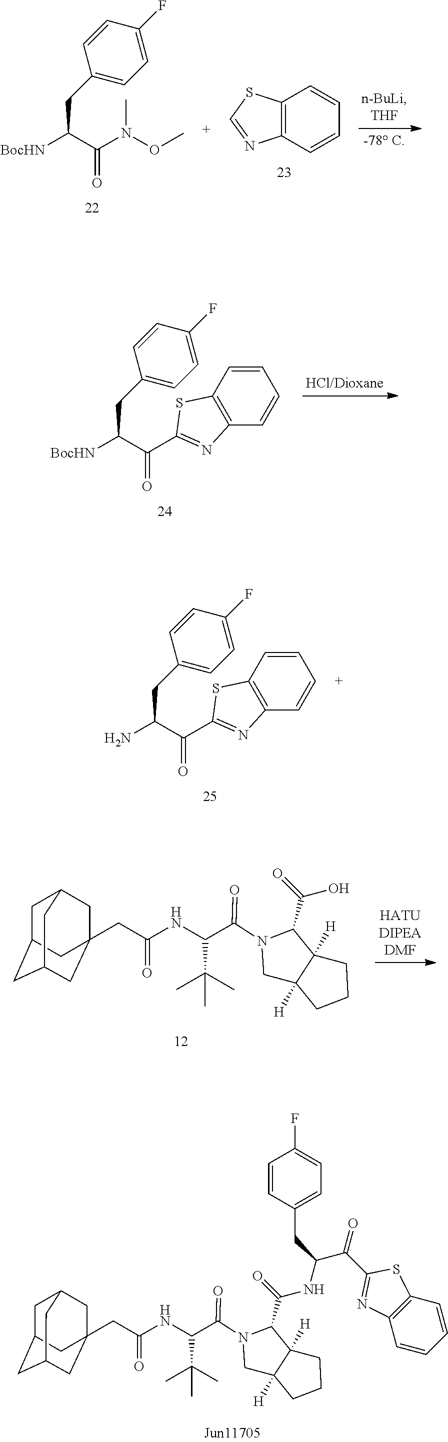

To a solution of benzothiazole 23 (5.4 g, 40 mmol) in 100 mL anhydrous tetrahydrofuran was added n-butyl lithium (2.5 M solution in hexane, 40 mmol) dropwise at −78° C. in 5 min with stirring under nitrogen. Upon completion of the addition, the reaction mixture was stirred for 30 min at −78° C. A solution of tert-butyl (S)-(3-(4-fluorophenyl)-1-(methoxy(methyl)amino)-1-oxopropan-2-yl)carbamate 22 (3.26 g, 10 mmol) in tetrahydrofuran (50 mL) was added at a rate to maintain the reaction temperature below −70° C. The resulting mixture was then slowly warmed to room temperature and quenched with saturated aqueous NH₄Cl (50 mL). The resulting organic layer was separated, washed with water. The organic extract was dried over Na₂SO₄, filtered, and concentrated under reduced pressure. The crude material was purified by chromatography to afford tert-butyl (S)-(1-(benzo[d]thiazol-2-yl)-3-(4-fluorophenyl)-1-oxopropan-2-yl)carbamate 24 (3.2 g, 80%).

To a solution of 24 (2.0 g, 5 mmol) in 20 mL dichloromethane was added 5 M, HCl in dioxane (4.0 mL, 20 mmol) The reaction mixture was stirred at room temperature for 2 h and the solvent was removed under reduced pressure to give crude (S)-2-amino-1-(benzo[d]thiazol-2-yl)-3-(4-fluorophenyl)propan-1-one 25. The resulting solid was used directly for the next step reaction without further purification. A mixture of substituted of carboxylic acid 12 (2.0 g, 4.6 mmol) and the resulting amine 25 (1.4 g, 4.6 mmol) in 10 mL DMF was added HATU (1.7 g, 4.6 mmol) followed by DIPEA (1.7 mL, 9.2 mmol). The reaction mixture was stirred at room temperature for 6 h, then diluted with water (30 mL) and extracted with EtOAc (40 mL). The organics were dried with Na₂SO₄ and concentrated in vacuo to give (1S,3aR,6aS)-2-((S)-2-(2-((3S,5S,7S)-adamantan-1-yl)acetamido)-3,3-dimethylbutanoyl)-N—((S)-4-(benzo[d]thiazol-2-yl)-1-(4-fluorophenyl)-3,4-dioxobutan-2-yl)octahydrocyclopenta[c]pyrrole-1-carboxamide Jun11705 (2.8 g, 80%) which were purified by flash chromatography.

(S)-2-amino-1-(benzo[d]thiazol-2-yl)-3-(4-fluorophenyl)propan-1-one (25). White solid. Yield: 82%. ¹H NMR (400 MHz, MeOD) δ 8.22-8.08 (m, 1H), 8.06 (dd, J=7.3, 1.9 Hz, 1H), 7.57 (pd, J=7.2, 1.5 Hz, 2H), 7.21 (dd, J=8.4, 5.3 Hz, 2H), 6.93 (t, J=8.7 Hz, 2H), 5.44 (dd, J=8.0, 5.4 Hz, 1H), 3.59-3.43 (m, 1H), 3.26 (dd, J=14.5, 8.0 Hz, 1H). ¹³C NMR (101 MHz, MeOD) δ 189.47, 162.46, 153.25, 137.18, 131.17, 131.09, 128.64, 127.57, 125.41, 122.57, 115.53, 57.16, 47.41, 47.20, 46.98, 36.00. C₁₆H₁₄FN₂OS, LCMS calculated for m/z [M+H]⁺: 301.4 (calculated), 301.0 (found).

(1S,3aR,6aS)-2-((S)-2-(2-((3S,5S,7S)-adamantan-1-yl)acetamido)-3,3-dimethylbutanoyl)-N—((R)-4-(benzo[d]thiazol-2-yl)-1-(4-fluorophenyl)-3,4-dioxobutan-2-yl)octahydrocyclo-penta[c]pyrrole-1-carboxamide (Jun11705). White solid. Yield: 81%. ¹H NMR (400 MHz, CDCl₃) δ 8.21 (t, J=8.3 Hz, 1H), 8.00 (d, J=7.9 Hz, 1H), 7.64-7.49 (m, 2H), 7.11-7.07 (m, 2H), 6.92 (dt, J=13.9, 8.6 Hz, 2H), 6.16-5.95 (m, 2H), 4.71 (d, J=9.6 Hz, 1H), 4.43 (dd, J=11.1, 2.8 Hz, 1H), 3.76 (d, J=5.8 Hz, 2H), 3.44 (ddd, J=26.5, 14.1, 5.5 Hz, 1H), 3.24 (ddd, J=43.2, 14.2, 6.9 Hz, 1H), 2.98-2.84 (m, 1H), 2.77 (dddd, J=29.1, 11.9, 6.8, 3.2 Hz, 2H), 1.97 (d, J=7.7 Hz, 5H), 1.89-1.81 (m, 2H), 1.70 (d, J=12.1 Hz, 3H), 1.61 (d, J=16.4 Hz, 8H), 1.51-1.46 (m, 1H), 1.41-1.36 (m, 1H), 1.26 (d, J=3.1 Hz, 2H), 0.99 (s, 9H), 0.87 (d, J=7.0 Hz, 1H). ¹³C NMR (101 MHz, CDCl₃) δ 192.30, 191.90, 171.76, 171.10, 170.75, 170.66, 163.72, 153.54, 137.27, 131.05, 128.15, 127.24, 127.21, 125.86, 122.40, 115.24, 66.33, 56.38, 54.39, 51.79, 46.04, 43.03, 42.65, 36.76, 35.27, 32.99, 32.38, 31.90, 28.66, 26.53, 25.43, 22.64, 14.10. C₄₂H₅₁FN₄₀₄S, HRMS calculated for m/z [M+H]⁺: 727.3693 (calculated), 727.3794 (found).

### Examples 19-34

Using procedures similar to those described herein, the following compounds were also prepared.

### Example 19

(1S)-2-((S)-2-(2-((3S,5S,7S)-adamantan-1-yl)acetamido)-3,3-dimethylbutanoyl)-N((S)-1-(4-(tert-butoxy)phenyl)-4-(cyclopropylamino)-3,4-dioxobutan-2 yl)octahydrocyclopenta[c]pyrrole-1-carboxamide (Jun 11671). White solid. Yield: 61%. ¹H NMR (400 MHz, CDCl₃) δ 7.01 (d, J=8.4 Hz, 2H), 6.96 (dd, J=12.8, 5.4 Hz, 2H), 6.87 (d, J=8.3 Hz, 2H), 6.16 (d, J=9.5 Hz, 1H), 5.52 (q, J=6.4 Hz, 1H), 4.69 (d, J=9.6 Hz, 1H), 4.41 (d, J=3.0 Hz, 1H), 3.79-3.67 (m, 2H), 3.24-3.10 (m, 2H), 2.82-2.71 (m, 3H), 1.97 (s, 5H), 1.90-1.80 (m, 2H), 1.71-1.56 (m, 16H), 1.32 (s, 8H), 1.00 (s, 2H), 0.97 (s, 8H), 0.83 (d, J=6.5 Hz, 2H), 0.62-0.55 (m, 2H). ¹³C NMR (101 MHz, CDCl₃) δ 195.33, 171.42, 170.98, 170.72, 160.42, 154.48, 130.26, 129.71, 124.09, 124.07, 78.27, 66.03, 56.32, 55.27, 54.27, 51.62, 45.89, 42.93, 42.60, 36.70, 36.66, 35.17, 32.92, 32.25, 31.76, 28.81, 28.59, 26.52, 25.36, 22.41, 6.45, 6.38. C₄₃H₆₂N₄O₆, HRMS calculated for m/z [M+H]⁺: 731.4748 (calculated), 731.4737 (found).

### Example 20

(1S)-2-((S)-2-(2-((3S,5S,7S)-adamantan-1-yl)acetamido)-3,3-dimethylbutanoyl)-N-((2S)-4-(cyclopropylamino)-3-hydroxy-4-oxo-1-(thiophen-3-yl)butan-2 yl)octahydrocyclopenta[c]pyrrole-1-carboxamide (Jun11818). White solid. Yield: 63%. ¹H NMR (400 MHz, CDCl₃) δ 7.11 (d, J=5.1 Hz, 1H), 6.93-6.86 (m, 3H), 6.82 (s, 1H), 6.11 (d, J=9.6 Hz, 1H), 5.50 (q, J=5.8 Hz, 1H), 4.70 (d, J=9.6 Hz, 1H), 4.39 (d, J=2.9 Hz, 1H), 3.79-3.73 (m, 2H), 3.59 (dd, J=15.3, 5.8 Hz, 1H), 3.50-3.46 (m, 1H), 2.84 (dd, J=9.6, 4.1 Hz, 1H), 2.80-2.74 (m, 2H), 1.96 (s, 2H), 1.95-1.93 (m, 3H), 1.90-1.83 (m, 2H), 1.70-1.6 (m, 4H), 1.59 (dd, J=13.0, 2.8 Hz, 9H), 1.52-1.35 (m, 3H), 0.97 (s, 9H), 0.85 (q, J=7.2, 6.7 Hz, 2H), 0.60 (t, J=4.4 Hz, 2H). ¹³C NMR (101 MHz, CDCl₃) δ 194.75, 171.62, 171.05, 170.83, 160.24, 137.15, 127.24, 127.02, 124.82, 66.26, 60.37, 56.37, 55.54, 54.34, 53.41, 51.70, 45.99, 42.97, 42.65, 36.74, 35.21, 32.98, 32.31, 31.77, 31.49, 28.64, 26.75, 26.55, 26.51, 25.38, 22.45, 14.18, 6.55, 6.44. C₃₇H₅₂N₄O₅S, HRMS calculated for m/z [M+H]⁺: 665.3737 (calculated), 665.3740 (found).

### Example 21

(1S,3aR,6aS)-2-((S)-2-(2-((3S,5S,7S)-adamantan-1-yl)acetamido)-3,3-dimethylbutanoyl)-N—((S)-4-(cyclopropylamino)-1-(1H-indol-3-yl)-3,4-dioxobutan-2-yl)octahydrocyclopenta[c]pyrrole-1-carboxamide (Jun 11862). White solid. Yield: 51%. ¹H NMR (400 MHz, CDCl₃) δ 8.47 (s, 1H), 7.50 (d, J=7.9 Hz, 1H), 7.30 (d, J=8.0 Hz, 1H), 7.15 (d, J=10.2 Hz, 2H), 7.06 (d, J=7.5 Hz, 1H), 6.99 (d, J=6.6 Hz, 1H), 6.83 (d, J=3.9 Hz, 1H), 6.11 (d, J=9.6 Hz, 1H), 5.63 (q, J=6.1 Hz, 1H), 4.65 (d, J=9.6 Hz, 1H), 4.39 (d, J=2.8 Hz, 1H), 3.72-3.67 (m, 2H), 3.41 (dd, J=5.9, 2.6 Hz, 2H), 2.80-2.71 (m, 4H), 1.94 (d, J=5.5 Hz, 8H), 1.59 (d, J=11.8 Hz, 11H), 0.88 (s, 10H), 0.82 (d, J=5.2 Hz, 2H), 0.53 (d, J=4.1 Hz, 2H). ¹³C NMR (101 MHz, DMSO-d₆) δ 195.47, 171.40, 171.03, 170.83, 160.55, 136.15, 127.32, 123.58, 122.12, 119.52, 118.55, 111.23, 109.25, 66.04, 56.40, 54.80, 54.39, 51.68, 45.90, 42.99, 42.64, 36.73, 35.14, 32.96, 32.34, 31.87, 28.63, 27.06, 26.53, 26.39, 25.41, 22.38, 6.42, 6.30. C₄₁H₅₅N₅O₅, HRMS calculated for m/z [M+H]⁺: 698.4281 (calculated), 698.4259 (found).

### Example 22

(1S,3aR,6aS)-2-((S)-2-(2-((3S,5S,7S)-adamantan-1-yl)acetamido)-3,3-dimethylbutanoyl)-N—((S)-4-(cyclopropylamino)-1-methoxy-3,4-dioxobutan-2-yl)octahydrocyclopenta[c]pyrrole-1-carboxamide (Jun 11672). White solid. Yield: 63%. ¹H NMR (400 MHz, CDCl₃) δ 7.36 (dd, J=18.7, 7.5 Hz, 1H), 7.01 (dd, J=21.2, 4.0 Hz, 1H), 6.19 (d, J=9.6 Hz, 1H), 5.47-5.41 (m, 1H), 4.74 (t, J=9.1 Hz, 1H), 4.52 (dd, J=13.0, 2.8 Hz, 1H), 4.17-4.11 (m, 1H), 3.82-3.69 (m, 3H), 3.26 (d, J=6.8 Hz, 3H), 2.97 (td, J=8.1, 4.1 Hz, 1H), 2.82-2.75 (m, 2H), 1.95 (d, J=4.1 Hz, 6H), 1.88 (dd, J=12.6, 7.1 Hz, 2H), 1.68 (s, 3H), 1.60 (d, J=12.7 Hz, 10H), 1.52-1.39 (m, 3H), 1.02 (d, J=6.3 Hz, 9H), 0.85 (d, J=5.8 Hz, 2H), 0.61 (d, J=3.3 Hz, 2H). ¹³C NMR (101 MHz, CDCl₃) δ 193.60, 193.25, 171.66, 171.40, 170.98, 170.91, 170.83, 170.79, 160.29, 160.27, 71.88, 71.86, 65.80, 59.15, 59.11, 56.36, 56.34, 55.53, 55.45, 54.28, 54.20, 51.66, 45.02, 44.99, 42.95, 42.83, 42.61, 36.70, 35.34, 35.29, 32.94, 32.31, 32.24, 31.94, 31.88, 28.60, 26.45, 26.43, 25.50, 25.48, 22.37, 22.34, 6.41, 6.35. C₃₄H₅₂N₄O₆, HRMS calculated for m/z [M+H]⁺: 613.3965 (calculated), 613.3887 (found).

### Example 23

(1S,3aR,6aS)-2-((S)-2-(2-((3S,5S,7S)-adamantan-1-yl)acetamido)-3,3-dimethylbutanoyl)-N—((S)-1-cyclopropyl-4-(cyclopropylamino)-3,4-dioxobutan-2-yl)octahydrocyclopenta[c]pyrrole-1-carboxamide (Jun 11761). White solid. Yield: 53%. ¹H NMR (400 MHz, CDCl₃) δ 7.21 (d, J=6.7 Hz, 1H), 6.91 (s, 1H), 6.01 (d, J=9.6 Hz, 1H), 5.37 (q, J=6.3 Hz, 1H), 4.71 (d, J=9.5 Hz, 1H), 4.46 (d, J=2.6 Hz, 1H), 3.79-3.6 (m, 3H), 2.96 (d, J=8.1 Hz, 1H), 2.84-2.74 (m, 3H), 1.96 (s, 8H), 1.86 (ddd, J=26.9, 13.5, 6.7 Hz, 4H), 1.70 (d, J=12.8 Hz, 6H), 1.62 (s, 7H), 1.55 (s, 2H), 0.99 (s, 8H), 0.85 (d, J=7.5 Hz, 2H), 0.59 (dd, J=8.5, 3.7 Hz, 2H), 0.44 (d, J=8.1 Hz, 2H). ¹³C NMR (101 MHz, CDCl₃) δ 196.47, 171.42, 170.91, 170.78, 160.47, 65.99, 56.36, 54.85, 54.43, 53.42, 51.55, 45.43, 43.06, 42.64, 42.61, 36.75, 36.71, 35.27, 32.91, 32.30, 31.89, 28.60, 26.49, 25.51, 22.38, 7.48, 6.44, 6.36, 5.27, 4.60. C₃₆H₅₄N₄O₅, HRMS calculated for m/z [M+H]⁺: 623.4172 (calculated), 623.4183 (found).

### Example 24

(1S,3aR,6aS)-2-((S)-2-(2-((3S,5S,7S)-adamantan-1-yl)acetamido)-3,3-dimethyl-butanoyl)-N—((S)-1-(cyclopropylamino)-5-methyl-1,2-dioxohexan-3-yl)octahydro-cyclopenta[c]pyrrole-1-carboxamide (Jun11389). White solid. Yield: 80%. ¹H NMR (400 MHz, CDCl₃) δ 7.04 (d, J=7.0 Hz, 1H), 6.91 (d, J=3.9 Hz, 1H), 6.03 (d, J=9.6 Hz, 1H), 5.30 (s, 1H), 4.72 (d, J=9.5 Hz, 1H), 4.44 (d, J=2.5 Hz, 1H), 3.81-3.61 (m, 2H), 2.96 (q, J=7.8 Hz, 1H), 2.79 (ddq, J=14.9, 7.4, 4.0 Hz, 2H), 1.95 (s, 4H), 1.91-1.85 (m, 2H), 1.69 (dt, J=13.0, 6.2 Hz, 7H), 1.62 (s, 4H), 1.58 (d, J=4.3 Hz, 4H), 1.56-1.44 (m, 4H), 1.38 (dt, J=20.6, 8.1 Hz, 2H), 0.99 (d, J=7.8 Hz, 11H), 0.92 (d, J=5.7 Hz, 3H), 0.89-0.87 (m, 1H), 0.87-0.81 (m, 3H), 0.59 (dt, J=11.5, 6.0 Hz, 2H). ¹³C NMR (101 MHz, CDCl₃) δ 196.34, 171.66, 170.93, 170.83, 160.54, 65.99, 56.40, 54.42, 52.90, 51.80, 44.74, 43.08, 42.72, 40.47, 36.79, 35.31, 33.04, 32.33, 28.70, 26.49, 25.57, 25.17, 23.28, 22.43, 21.34, 6.43. C₃₆H₅₆N₄O₅, HRMS calculated for m/z [M+Na]⁺: 647.4148 (calculated), 647.3351 (found).

### Example 25

(1S,3aR,6aS)-2-((S)-2-(2-((3S,5S,7S)-adamantan-1-yl)acetamido)-3,3-dimethylbutanoyl)-N—((S)-3-(cyclopropylamino)-2,3-dioxo-1-phenylpropyl)octahydrocyclopenta[c]pyrrole-1-carboxamide (Jun 11817). White solid. Yield: 62%. ¹H NMR (400 MHz, CDCl₃) δ 7.73 (d, J=6.5 Hz, 1H), 7.37 (d, J=8.6 Hz, 3H), 7.30 (s, 2H), 7.03 (d, J=4.0 Hz, 1H), 6.42 (d, J=6.4 Hz, 2H), 6.21 (d, J=9.6 Hz, 1H), 4.66 (d, J=9.5 Hz, 1H), 4.57 (d, J=2.6 Hz, 1H), 3.77-3.71 (m, 2H), 3.64 (dd, J=10.8, 7.9 Hz, 1H), 3.03-2.96 (m, 2H), 2.83-2.75 (m, 2H), 2.70-2.65 (m, 2H), 1.95 (s, 7H), 1.67 (s, 5H), 1.05 (s, 5H), 0.80 (s, 10H), 0.60-0.53 (m, 2H), 0.51-0.45 (m, 2H). ¹³C NMR (101 MHz, CDCl₃) δ 192.63, 192.26, 171.85, 171.66, 170.87, 170.35, 170.24, 159.97, 133.78, 133.68, 129.11, 129.08, 128.72, 128.66, 128.46, 128.43, 65.76, 65.72, 57.94, 57.92, 56.41, 56.29, 54.32, 51.57, 51.54, 44.35, 44.32, 42.93, 42.91, 42.60, 36.70, 35.30, 35.10, 32.97, 32.94, 32.30, 32.24, 32.03, 31.92, 28.60, 26.51, 26.30, 25.58, 25.55, 22.42, 22.39, 6.35, 6.31. C₃₈H₅₂N₄O₅, HRMS calculated for m/z [M+H]⁺: 645.4016 (calculated), 645.4038 (found).

### Example 26

(1S,3aR,6aS)-2-((S)-2-(2-((3S,5S,7S)-adamantan-1-yl)acetamido)-3,3-dimethylbutanoyl)-N—((S)-3-(cyclopropylamino)-1-(4-fluorophenyl)-2,3-dioxopropyl)octahydrocyclopenta[c]pyrrole-1-carboxamide (Jun11861). White solid. Yield: 71%. ¹H NMR (400 MHz, CDCl₃) δ 7.79 (dd, J=17.4, 6.4 Hz, 1H), 7.40-7.35 (m, 2H), 7.01 (d, J=8.9 Hz, 2H), 6.40 (t, J=6.8 Hz, 1H), 6.19 (dd, J=27.0, 9.5 Hz, 1H), 4.70 (dd, J=40.8, 9.5 Hz, 1H), 4.51 (dd, J=23.9, 2.7 Hz, 1H), 3.73 (dddd, J=45.0, 26.4, 10.8, 5.9 Hz, 3H), 3.02-2.94 (m, 1H), 2.83-2.75 (m, 1H), 2.72-2.66 (m, 1H), 1.97 (d, J=8.0 Hz, 6H), 1.86 (dt, J=14.3, 7.1 Hz, 2H), 1.70-1.57 (m, 16H), 1.43 (ddt, J=33.1, 13.0, 6.6 Hz, 3H), 1.05 (s, 3H), 0.82 (s, 4H), 0.59-0.48 (m, 2H). ¹³C NMR (101 MHz, CDCl₃) δ 192.41, 192.09, 171.93, 171.74, 170.91, 170.38, 170.29, 159.90, 159.84, 130.36, 130.28, 129.69, 116.20, 115.99, 65.80, 57.23, 57.19, 56.42, 56.30, 54.34, 51.62, 51.59, 44.32, 42.96, 42.92, 42.61, 36.69, 35.29, 35.09, 32.98, 32.96, 32.28, 32.22, 31.98, 31.88, 28.60, 26.50, 26.29, 25.56, 25.52, 22.45, 22.42, 6.39, 6.36. C₃₈H₅₁FN₄O₅, HRMS calculated for m/z [M+H]⁺: 663.3922 (calculated), 663.3927 (found).

### Example 27

(1S,3aR,6aS)-2-((S)-2-(2-((3S,5S,7S)-adamantan-1-yl)acetamido)-3,3-dimethylbutanoyl)-N—((S)-1-cyclohexyl-3-(cyclopropylamino)-2,3-dioxopropyl)octahydrocyclopenta[c]pyrrole-1-carboxamide (Jun 11762). White solid. Yield: 40%. ¹H NMR (400 MHz, DMSO-d₆) δ 8.69 (d, J=5.1 Hz, 1H), 8.11 (d, J=7.5 Hz, 1H), 7.63 (d, J=9.2 Hz, 1H), 4.96 (t, J=6.6 Hz, 1H), 4.51 (d, J=9.1 Hz, 1H), 4.33 (d, J=3.5 Hz, 1H), 4.03 (q, J=7.1 Hz, 1H), 3.72 (d, J=7.1 Hz, 2H), 2.74 (td, J=7.4, 3.7 Hz, 1H), 2.63 (tq, J=7.8, 4.0, 3.2 Hz, 1H), 1.88 (s, 5H), 1.66-1.53 (m, 19H), 1.19-1.04 (m, 8H), 0.92 (s, 11H), 0.65 (d, J=6.3 Hz, 2H), 0.56 (d, J=3.6 Hz, 2H). ¹³C NMR (101 MHz, DMSO-d₆) δ 197.63, 172.44, 170.77, 170.74, 170.16, 162.91, 65.10, 60.20, 58.22, 56.42, 54.45, 49.48, 47.43, 42.84, 42.49, 39.25, 36.94, 34.74, 32.97, 32.31, 32.11, 29.72, 28.58, 28.24, 26.91, 26.15, 26.05, 25.98, 25.14, 22.97, 21.20, 14.54, 5.89, 5.85. C₃₈H₅₈N₄O₅, HRMS calculated for m/z [M+H]⁺: 651.4485 (calculated), 651.4490 (found).

### Example 28

(1S,3aR,6aS)-2-((S)-2-(2-((3S,5S,7S)-adamantan-1-yl)acetamido)-3,3-dimethylbutanoyl)-N—((S)-3-amino-1-cyclohexyl-2,3-dioxopropyl)octahydro-cyclopenta[c]pyrrole-1-carboxamide (Jun 11725). White solid. Yield: 62%. ¹H NMR (400 MHz, CDCl₃) δ 7.33 (d, J=7.5 Hz, 1H), 6.94 (d, J=3.1 Hz, 1H), 6.55 (d, J=3.3 Hz, 1H), 6.38 (d, J=9.7 Hz, 1H), 5.17 (t, J=6.7 Hz, 1H), 4.75 (d, J=9.6 Hz, 2H), 4.46 (d, J=2.7 Hz, 1H), 3.80 (dt, J=10.8, 3.8 Hz, 2H), 3.75-3.68 (m, 2H), 2.96-2.92 (m, 1H), 2.82 (ddt, J=12.1, 7.9, 4.3 Hz, 2H), 1.96 (s, 9H), 1.91-1.82 (m, 5H), 1.69 (d, J=12.5 Hz, 12H), 1.02 (s, 13H). ¹³C NMR (101 MHz, CDCl₃) δ 196.19, 171.73, 171.06, 170.95, 161.56, 66.05, 58.40, 56.37, 54.45, 51.53, 44.82, 43.00, 42.63, 39.91, 36.73, 35.26, 32.98, 32.22, 31.87, 30.03, 28.63, 28.29, 26.47, 25.88, 25.80, 25.75, 25.49. C₃₅H₅₄N₄O₅, HRMS calculated for m/z [M+H]⁺: 611.4172 (calculated), 611.4176 (found).

### Example 29

(1S,3aR,6aS)-2-((S)-2-(2-((3S,5S,7S)-adamantan-1-yl)acetamido)-3,3-dimethylbutanoyl)-N—((S)-1-cyclopentyl-3-(cyclopropylamino)-2,3-dioxopropyl)octahydrocyclopenta[c]pyrrole-1-carboxamide (Jun 11808). White solid. Yield: 57%. ¹H NMR (400 MHz, CDCl₃) δ 7.43 (d, J=7.0 Hz, 1H), 7.16 (d, J=4.2 Hz, 1H), 6.33 (d, J=9.6 Hz, 1H), 5.15 (t, J=7.2 Hz, 1H), 4.76 (d, J=9.5 Hz, 1H), 4.50 (d, J=2.0 Hz, 1H), 3.78 (d, J=5.1 Hz, 2H), 2.82 (d, J=3.7 Hz, 3H), 2.35 (q, J=8.2 Hz, 1H), 2.05-1.96 (m, 5H), 1.85 (dt, J=13.7, 7.0 Hz, 2H), 1.71-1.56 (m, 18H), 1.54-1.46 (m, 4H), 1.36 (dt, J=13.0, 6.4 Hz, 2H), 1.30-1.23 (m, 1H), 1.01 (s, 8H), 0.84 (d, J=7.6 Hz, 2H), 0.62 (d, J=8.9 Hz, 2H). ¹³C NMR (101 MHz, CDCl₃) δ 196.09, 171.41, 171.20, 170.80, 160.91, 65.74, 57.04, 56.31, 54.46, 51.46, 45.37, 42.93, 42.60, 41.44, 36.69, 35.22, 32.91, 32.20, 31.83, 28.92, 28.83, 28.58, 26.48, 25.45, 24.95, 24.55, 22.50, 6.43, 6.30. C₃₇H₅₆N₄O₅, HRMS calculated for m/z [M+H]⁺: 637.4329 (calculated), 637.4333 (found).

### Example 30

(1S,3aR,6aS)-2-((S)-2-(2-((3S,5S,7S)-adamantan-1-yl)acetamido)-3,3-dimethylbutanoyl)-N—((S)-1-(cyclopropylamino)-4-methyl-1,2-dioxopentan-3-yl)octahydrocyclopenta[c]pyrrole-1-carboxamide (Jun 11718). White solid. Yield: 42%. ¹H NMR (400 MHz, CDCl₃) δ 7.19 (d, J=7.7 Hz, 1H), 6.94 (d, J=3.9 Hz, 1H), 6.14 (d, J=9.6 Hz, 1H), 5.24 (dd, J=7.6, 5.0 Hz, 1H), 4.74 (d, J=9.6 Hz, 1H), 4.45 (d, J=2.6 Hz, 1H), 3.77-3.75 (m, 2H), 2.90-2.85 (m, 1H), 2.84-2.75 (m, 2H), 2.40-2.32 (m, 1H), 1.96 (s, 4H), 1.86 (dd, J=13.3, 6.8 Hz, 2H), 1.68 (t, J=10.7 Hz, 6H), 1.63 (s, 5H), 1.59 (s, 4H), 1.56 (s, 1H), 1.53-1.34 (m, 3H), 1.01 (d, J=2.5 Hz, 10H), 0.85 (d, J=7.0 Hz, 5H), 0.60 (dt, J=6.7, 3.0 Hz, 2H). ¹³C NMR (101 MHz, CDCl₃) δ 196.38, 171.62, 171.26, 170.79, 160.60, 66.15, 58.80, 56.38, 54.44, 51.75, 45.25, 43.07, 42.69, 36.77, 35.34, 33.00, 32.25, 31.84, 30.21, 28.67, 26.55, 25.50, 22.49, 19.91, 17.53, 6.53, 6.46. C₃₅H₅₄N₄O₅, HRMS calculated for m/z [M+H]⁺: 611.4172 (calculated), 611.4175 (found).

### Example 31

(1S,3aR,6aS)-2-((S)-2-(2-((3S,5S,7S)-adamantan-1-yl)acetamido)-3,3-dimethylbutanoyl)-N-((3S,4S)-1-(cyclopropylamino)-4-methyl-1,2-dioxohexan-3-yl)octahydrocyclopenta[c]pyrrole-1-carboxamide (Jun11756). White solid. Yield: 74%. ¹H NMR (400 MHz, CDCl₃) δ 7.26 (d, J=7.7 Hz, 1H), 7.02 (d, J=4.0 Hz, 1H), 6.20 (d, J=9.6 Hz, 1H), 5.24 (dd, J=7.7, 5.5 Hz, 1H), 4.74 (d, J=9.7 Hz, 1H), 4.46 (d, J=2.6 Hz, 1H), 3.76 (d, J=6.9 Hz, 2H), 2.91-2.76 (m, 4H), 2.13-2.05 (m, 1H), 1.97 (s, 5H), 1.90-1.82 (m, 3H), 1.68 (t, J=11.6 Hz, 6H), 1.62 (s, 5H), 1.52-1.45 (m, 2H), 1.41-1.31 (m, 3H), 1.01 (s, 9H), 0.96 (d, J=6.8 Hz, 3H), 0.85 (t, J=7.3 Hz, 5H), 0.62-0.59 (m, 2H). ¹³C NMR (101 MHz, CDCl₃) δ 196.54, 171.53, 171.16, 170.75, 160.70, 66.03, 60.34, 58.34, 56.33, 54.41, 51.68, 51.63, 45.17, 43.01, 42.64, 42.62, 36.86, 36.71, 35.28, 32.95, 32.21, 31.83, 28.61, 26.48, 25.47, 24.52, 22.45, 16.05, 14.16, 14.10, 11.25, 6.48, 6.40. C₃₆H₅₆N₄O₅, HRMS calculated for m/z [M+H]⁺: 625.4329 (calculated), 625.4330 (found).

### Example 32

(1S,3aR,6aS)-2-((S)-2-(2-((3S,5S,7S)-adamantan-1-yl)acetamido)-3,3-dimethylbutanoyl)-N—((S)-1-(cyclopropylamino)-4,4-dimethyl-1,2-dioxopentan-3-yl)octahydrocyclopenta[c]pyrrole-1-carboxamide (Jun11724). White solid. Yield: 50%. ¹H NMR (400 MHz, CDCl₃) δ 7.52 (dd, J=32.5, 7.2 Hz, 1H), 6.98 (dd, J=51.0, 4.0 Hz, 1H), 6.16 (dd, J=30.5, 9.6 Hz, 1H), 5.37 (dd, J=7.2, 3.1 Hz, 1H), 4.74 (d, J=9.6 Hz, 1H), 4.48 (dd, J=5.4, 2.2 Hz, 1H), 3.76 (d, J=5.2 Hz, 1H), 2.85-2.73 (m, 3H), 1.96 (d, J=2.9 Hz, 5H), 1.89-1.82 (m, 2H), 1.71-1.56 (m, 14H), 1.51-1.31 (m, 3H), 1.03-0.98 (m, 18H), 0.84 (ddd, J=7.4, 4.6, 3.1 Hz, 2H), 0.65-0.53 (m, 2H). ¹³C NMR (101 MHz, CDCl₃) δ 197.56, 171.61, 170.93, 170.74, 161.21, 65.83, 65.62, 58.76, 58.68, 56.35, 56.33, 54.43, 54.25, 51.68, 51.61, 45.09, 44.21, 42.97, 42.90, 42.63, 42.61, 36.70, 35.22, 35.05, 34.77, 32.96, 32.94, 32.15, 32.08, 31.84, 31.71, 28.60, 26.67, 26.51, 26.43, 25.54, 25.44, 22.54, 22.46, 6.60, 6.56, 6.36, 6.32. C₃₆H₅₆N₄O₅, HRMS calculated for m/z [M+H]⁺: 625.4329 (calculated), 625.4425 (found).

### Example 33

(1S,3aR,6aS)-2-((S)-2-(2-((3S,5S,7S)-adamantan-1-yl)acetamido)-3,3-dimethylbutanoyl)-N-(1-(2-(cyclopropylamino)-2 oxoacetyl)cyclobutyl)octahydrocyclopenta[c]pyrrole-1-carboxamide (Jun 11806). White solid. Yield: 68%. ¹H NMR (400 MHz, CDCl₃) δ 8.33 (s, 1H), 6.95 (d, J=4.3 Hz, 1H), 6.46 (d, J=9.6 Hz, 1H), 4.75 (d, J=9.6 Hz, 1H), 4.39 (d, J=3.6 Hz, 1H), 3.87-3.77 (m, 2H), 2.97-2.90 (m, 1H), 2.79-2.73 (m, 2H), 2.71-2.65 (m, 1H), 2.61-2.53 (m, 1H), 2.43 (dt, J=12.3, 8.8 Hz, 1H), 2.15-2.07 (m, 2H), 1.98-1.95 (m, 5H), 1.84 (ddd, J=13.9, 8.3, 6.1 Hz, 3H), 1.70 (d, J=11.8 Hz, 4H), 1.64 (s, 8H), 1.60-1.57 (m, 2H), 1.57 (d, J=6.6 Hz, 1H), 1.46-1.40 (m, 2H), 1.02 (s, 9H), 0.79 (d, J=6.5 Hz, 2H), 0.58 (dd, J=15.8, 3.5 Hz, 2H). ¹³C NMR (101 MHz, CDCl₃) δ 195.15, 171.46, 171.27, 170.93, 162.13, 65.69, 61.03, 56.47, 54.48, 51.27, 46.26, 43.17, 42.59, 36.72, 35.27, 32.90, 32.02, 31.59, 31.32, 30.59, 28.60, 26.68, 25.33, 22.36, 14.74, 6.36, 6.22. C₃₅H₅₂N₄O₅, HRMS calculated for m/z [M+H]⁺: 609.4016 (calculated), 609.4020 (found).

### Example 34

(1S,3aR,6aS)-2-((S)-2-(2-((3S,5S,7S)-adamantan-1-yl)acetamido)-3,3-dimethylbutanoyl)-N-(1-(2-(cyclopropylamino)-2-oxoacetyl)cyclohexyl)octahydrocyclopenta[c]pyrrole-1-carboxamide (Jun 11805). White solid. Yield: 51%. ¹H NMR (400 MHz, CDCl₃) δ 7.43 (s, 1H), 6.76 (d, J=3.8 Hz, 1H), 6.13 (d, J=9.6 Hz, 1H), 4.75 (d, J=9.6 Hz, 1H), 4.46 (d, J=2.6 Hz, 1H), 3.76 (d, J=6.2 Hz, 2H), 2.93-2.86 (m, 1H), 2.82-2.76 (m, 1H), 2.70-2.64 (m, 1H), 2.18 (s, 1H), 1.97-1.95 (m, 5H), 1.91-1.78 (m, 5H), 1.70 (d, J=12.9 Hz, 8H), 1.63-1.55 (m, 8H), 1.53-1.45 (m, 4H), 1.38 (dd, J=13.0, 6.6 Hz, 1H), 1.03 (s, 10H), 0.77 (d, J=5.5 Hz, 2H), 0.54 (d, J=3.7 Hz, 2H). ¹³C NMR (101 MHz, CDCl₃) δ 198.10, 171.76, 170.97, 170.72, 162.69, 65.90, 61.40, 56.34, 54.34, 51.65, 44.61, 42.90, 42.62, 36.71, 35.25, 32.96, 32.18, 31.83, 31.72, 31.33, 28.61, 26.57, 26.46, 25.49, 24.93, 22.15, 21.41, 21.28, 6.34, 6.24. C₃₇H₅₆N₄O₅, HRMS calculated for m/z [M+H]⁺: 637.4329 (calculated), 637.4334 (found).

### Example 35

The following illustrate representative pharmaceutical dosage forms, containing a compound of formula I (‘Compound X’), for therapeutic or prophylactic use in humans.

| i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |

| vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula (I): 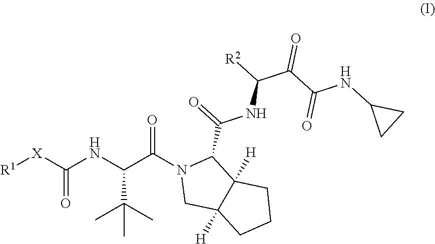
or a salt thereof wherein:
X is a bond or is (C₁-C₆)alkylene;
R¹ is (C₃-C₁₅)cycloalkyl that is optionally substituted with one or more groups independently selected from the group consisting of halo and (C₁-C₆)alkyl, which (C₁-C₆)alkyl is optionally substituted with one or more groups independently selected from the group consisting of halo; and
R² is (5-10 membered heteroaryl)(C₁-C₃)alkyl, cyclopropylmethyl, 2-methylpropyl, (C₃-C₆)cycloalkyl, isopropyl, propyl, or 1-methylpropyl.

2. The compound of claim 1, wherein R¹ is (C₆-C₁₀)cycloalkyl that is optionally substituted with one or more groups independently selected from the group consisting of halo and (C₁-C₆)alkyl, which (C₁-C₆)alkyl is optionally substituted with one or more groups independently selected from the group consisting of halo.

3. The compound or salt of claim 1, wherein R¹ is (C₆-C₁₀)cycloalkyl.

4. The compound or salt of claim 1, wherein R¹ is adamantyl that is optionally substituted with one or more groups independently selected from the group consisting of halo and (C₁-C₆)alkyl, which (C₁-C₆)alkyl is optionally substituted with one or more groups independently selected from the group consisting of halo.

5. The compound or salt of claim 1, wherein R¹ is adamantyl.

6. The compound or salt of claim 1, wherein R² is (5-membered heteroaryl)(C₁-C₃)alkyl.

7. The compound or salt of claim 1, wherein R² is (9-membered heteroaryl)(C₁-C₃)alkyl.

8. The compound or salt of claim 1, wherein R² is (5-membered heteroaryl)methyl.

9. The compound or salt of claim 1, wherein R² is (9-membered heteroaryl)methyl.

10. The compound or salt of claim 1, wherein R² is cyclopropylmethyl.

11. The compound or salt of claim 1, wherein R² is 2-methylpropyl.

12. The compound or salt of claim 1, wherein R² is (C₃-C₆)cycloalkyl.

13. The compound or salt of claim 1, wherein R² is cyclopentyl.

14. The compound or salt of claim 1, wherein R² is cyclohexyl.

15. The compound of or salt of claim 1, wherein R² is isopropyl.

16. The compound of or salt of claim 1, wherein R² is propyl.

17. The compound of or salt of claim 1, wherein R² is 1-methylpropyl.

18. A compound or salt selected from: 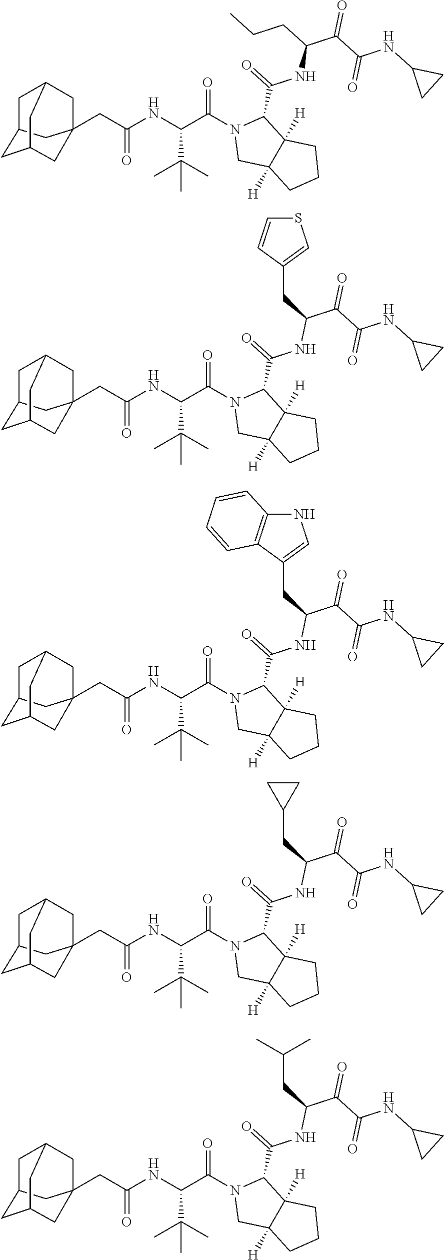 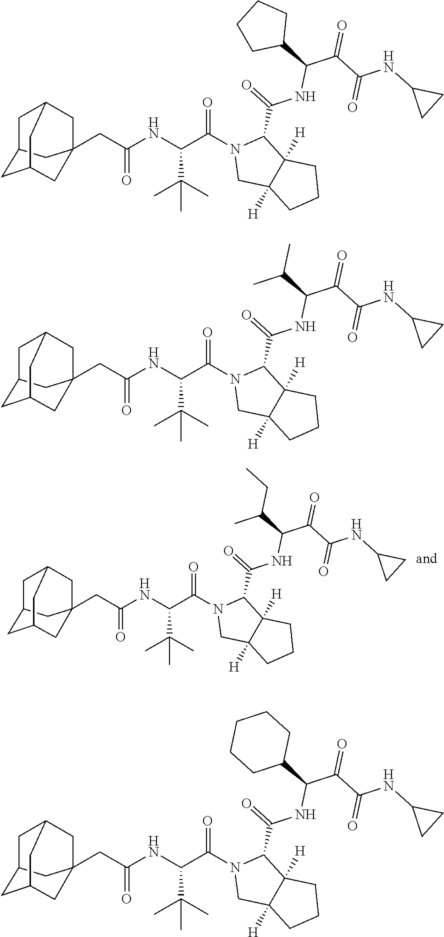
and salts thereof.

19. A pharmaceutical composition comprising a compound as described in claim 1 or a pharmaceutically acceptable salt thereof and a pharmaceutically acceptable excipient.

20. A method for treating or preventing a viral infection (EV-D68) in an animal comprising administering a compound as described in claim 1 or a pharmaceutically acceptable salt thereof to the animal.
